(19) United States
(12) Patent Application Publication
(10) Pub. No.: US 2014/0080848 A1
(43) Pub. Date: Mar. 20, 2014

(54) ARYL AMINE SUBSTITUTED PYRIMIDINE AND QUINAZOLINE AND THEIR USE AS ANTICANER DRUGS

(71) Applicants: National Taiwan University, Taipei (TW); National Yang-Ming University, Taipei City (TW)

(72) Inventors: Chung-Wai SHIAU, Taipei (TW); Kuen-Feng CHEN, Taipei (TW)

(73) Assignees: National Taiwan University, Taipei (TW); National Yang-Ming University, Taipei City (TW)

(21) Appl. No.: 14/026,545

(22) Filed: Sep. 13, 2013

Related U.S. Application Data
(60) Provisional application No. 61/700,923, filed on Sep. 14, 2012.

(51) Int. Cl. C07D 239/42; C07D 239/94; C07D 239/95; C07D 239/48

(52) U.S. Cl. CPC C07D 239/42; C07D 239/48; C07D 239/94; C07D 239/95
USPC 514/266.4; 514/256; 544/326; 514/275; 544/323; 544/293; 544/291; 435/375

(57) ABSTRACT

A series of mono- and di-substituted quinazoline and pyrimidine derivatives based on the skeleton of erlotinib (an EGFR inhibitor) were synthesized and their bioactivities against hepatocellular carcinoma and human lung adenocarcinoma were evaluated.

### BACKGROUND

1. Technical Field

The disclosure relates to pyrimidine and quinazoline derivatives and their use as anticancer drugs.

2. Description of Related Art

Overexpression of cancerous inhibitor of protein phosphatase 2A (abbreviated as CIP2A) has been found in several common human cancers including acute leukemia, prostate cancer, non-small cell lung cancer, gastric cancer, head and neck cancer, colon cancer and breast cancer and has been linked to clinical aggressiveness in tumors and promotion of the malignant growth of cancer cells. CIP2A interacts directly with the transcription factor c-Myc and inhibits the PP2A dephosphorylation of c-Myc, thereby stabilizing the oncogenic c-Myc from degradation.

Protein phosphatase 2A (abbreviated as PP2A) is a crucial regulator of cell proliferation by dephosphorylation of protein kinases on serine or threonine residues. PP2A is composed of three subunits which regulate substrate specificity, cellular localization and enzymatic activity. For example, PP2A dephosphorylates p-Akt at serine 473 and reduces the cell growth. Hence, the CIP2A-PP2A-Akt signaling cascade is thought to be an important survival regulator in cancers. Accordingly, downregulation of c-Myc and p-Akt by CIP2A ablation is a promising anticancer strategy.

Some compounds have been found to be capable of repressing repress CIP2A expression and subsequently reducing p-Akt level and induce apoptosis in hepatocellular carcinoma (HCC). For example, the above phenomenon had been observed for bortezomib, a proteasome inhibitor.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present invention is directed to an aryl amine substituted pyrimidine having a chemical structure (I) or (II) below:

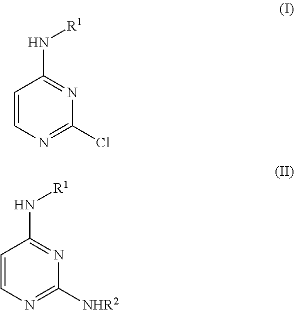

The R¹ and R² above are same or different substituted phenyl groups, and the substituted phenyl group each is

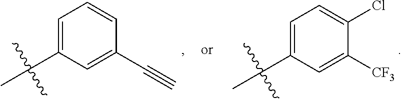

In another aspect, the present invention is directed to an aryl amine substituted quinazoline having a chemical structure (III) or (IV) below:

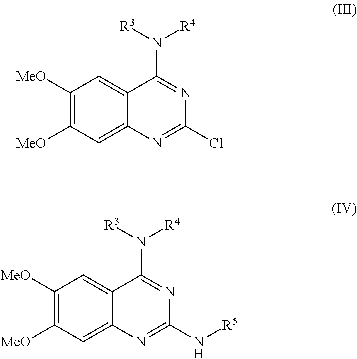

The R³ above is an aliphatic-substituted phenyl group, a halo-substituted phenyl group, a hydroxyl-substituted phenyl group, or an aryloxy-substituted phenyl group. The R4 above is H, an aliphatic group with carbon number of 1-5, an amino-substituted aliphatic group, or a benzyl group. The R⁵ above aliphatic substituted phenyl group, a halo-substituted phenyl group, an aryloxy-substituted phenyl group, a benzyl group, a halo substituted benzyl group, an alkoxy substituted phenyl group, an arylamino-substituted phenyl group, an amidyl-substituted phenyl group, an ArO(CO)NH-substituted phenyl group, or Ph-SO₂—NH-substituted phenyl group.

According to an embodiment, the R³ is

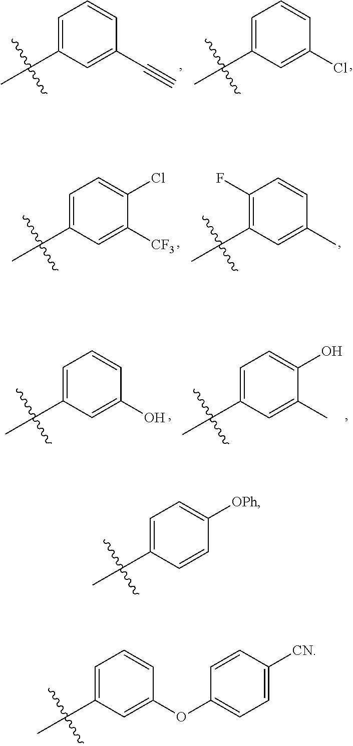

According to another embodiment, the R⁴ is H. Me

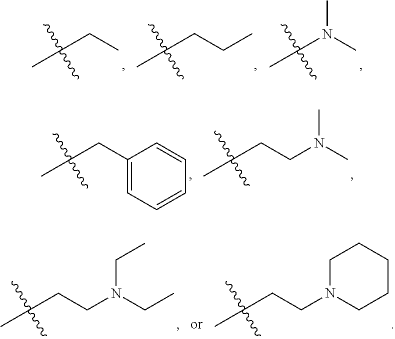

According to yet another embodiment, the R⁵ is

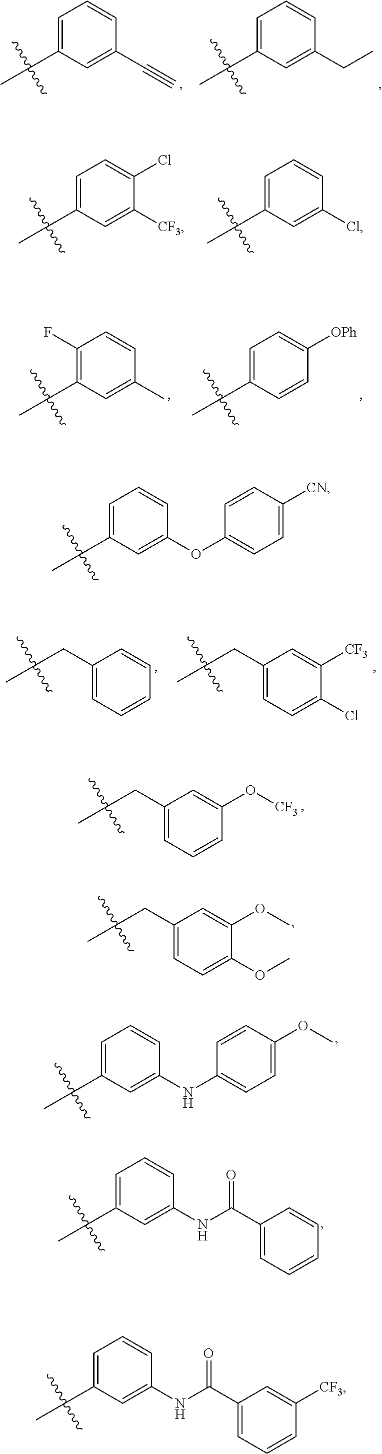

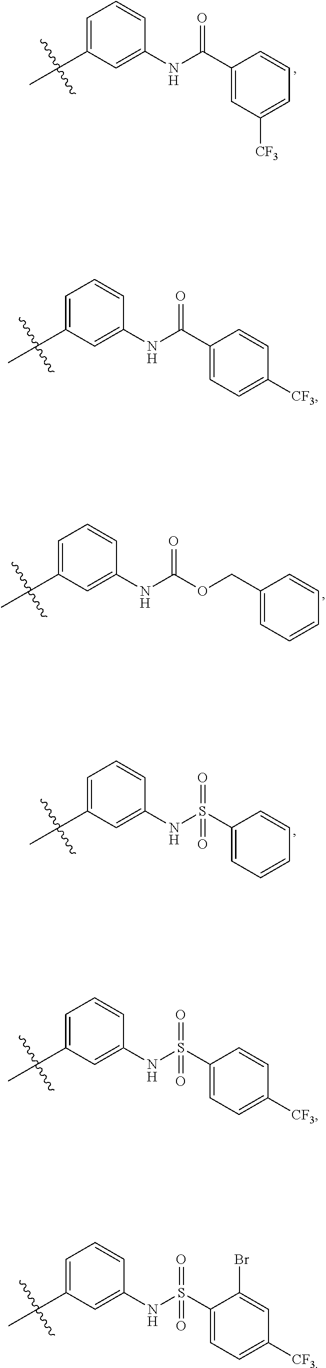 [SMILES: *c1cccc(NC(=O)c2cccc(C(F)(F)F)c2)c1 ; *c1cccc(NC(=O)c2ccc(C(F)(F)F)cc2)c1 ; *c1cccc(NC(=O)OCc2ccccc2)c1 ; *c1cccc(NS(=O)(=O)c2ccccc2)c1 ; *c1cccc(NS(=O)(=O)c2ccc(C(F)(F)F)cc2)c1 ; *c1cccc(NS(=O)(=O)c2ccc(C(F)(F)F)cc2Br)c1]

In yet another aspect, the present invention is directed to a method of synthesizing the aryl amine substituted pyrimidine having the chemical structure (I) or (II) above. The method comprises the following steps. First, 2,4-dichloropyrimidine reacts with a first substituted phenyl amine to form the compound of the chemical structure (I), wherein the first substituted phenyl amine having a first substituted phenyl group of

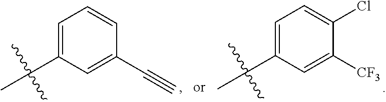

Then, the compound of the chemical structure (I) reacts with a second substituted phenyl amine to form the compound of the chemical structure (II), wherein the second substituted phenyl amine having a second substituted phenyl group of

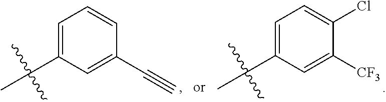

In yet another aspect, the present invention is directed to a method of synthesizing the aryl amine substituted quinazoline having the chemical structure (III) or (IV) above. For the compound having the chemical structure (III), the method comprises the following steps. 2,4-dichloroquinazoline reacts with a substituted phenyl amine, wherein the substituted phenyl amine having a substituted phenyl group of

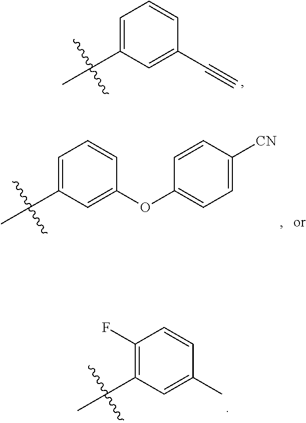

For the compound having the chemical structure (IV), 2,4-dichloroquinazoline reacts with R³R⁴NH to obtain

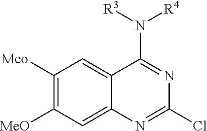

first. Then,

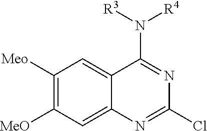

reacts with a R⁵NH₂.

In yet another aspect, the present invention directs to a pharmaceutical composition. The pharmaceutical composition comprises an effective amount of a compound having a chemical structure (I), (II), (III), or (VII) below and a pharmaceutically acceptable carrier.

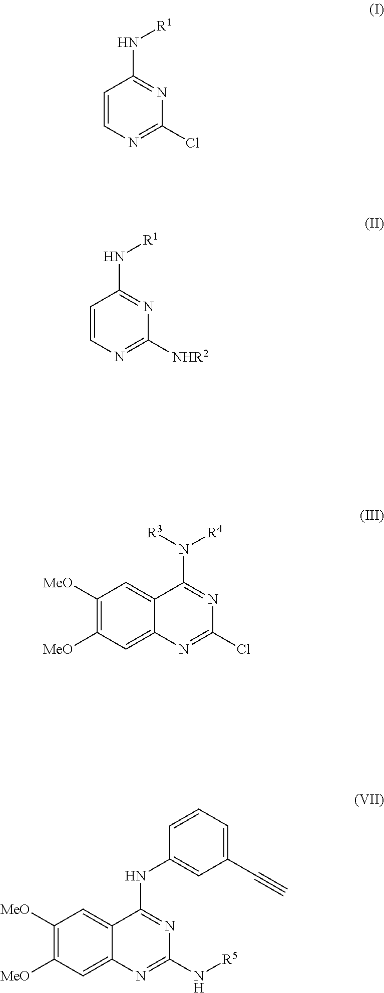

The R³ above is

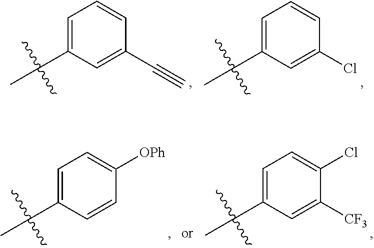

and R⁴ is H or methyl group. The R⁵ above is

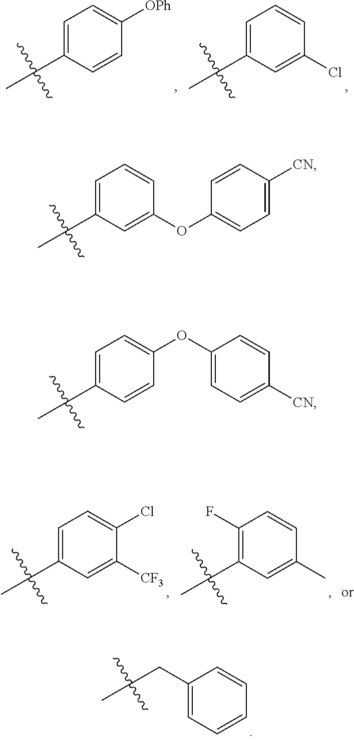

In yet another aspect, the present invention directs to a method of inhibiting the expression of cancerous inhibitor of PP2A. The method comprises contacting a cell with an effective amount of a compound having a chemical structure (I), (II), (III), or (VII) above. The R³ above is

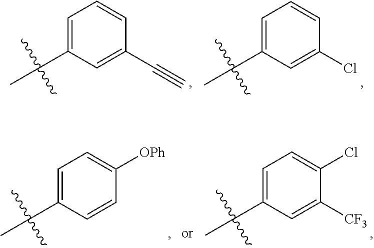

and R⁴ is H or methyl group. The R⁵ above is

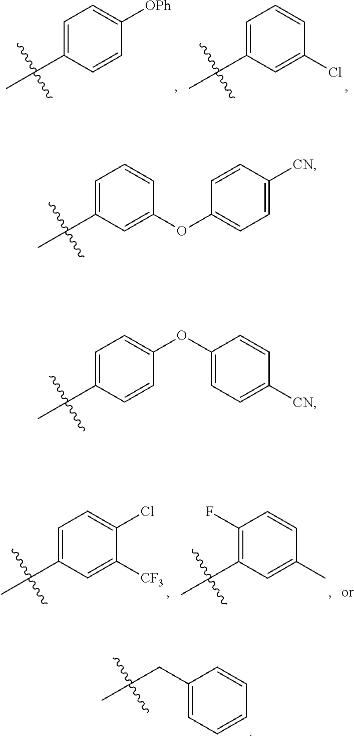

In yet another aspect, the present invention directs to a method of treating cancer. The method comprises administrating an effective amount of a compound having a chemical structure (I), (II), (III), or (VII) above by a needed subject. The R³ above is

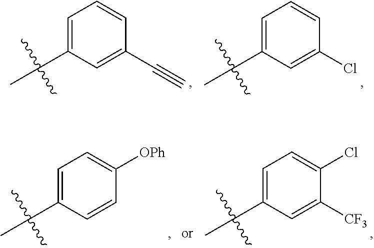

and R⁴ is H or methyl group. The R⁵ above is

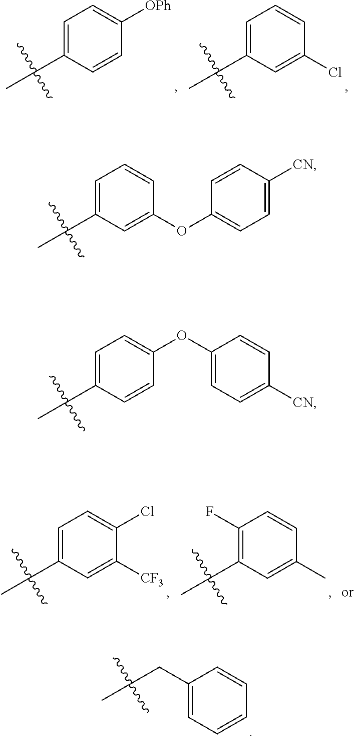

In yet another aspect, the present invention directs to a pharmaceutical composition. The pharmaceutical composition comprises an effective amount of a compound having a chemical structure (I), (II), (III), or (IV) above and a pharmaceutically acceptable carrier. The R³ above is

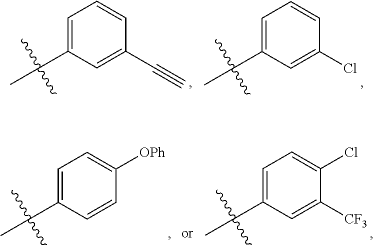

and R⁴ is H or methyl group. The R⁵ above is

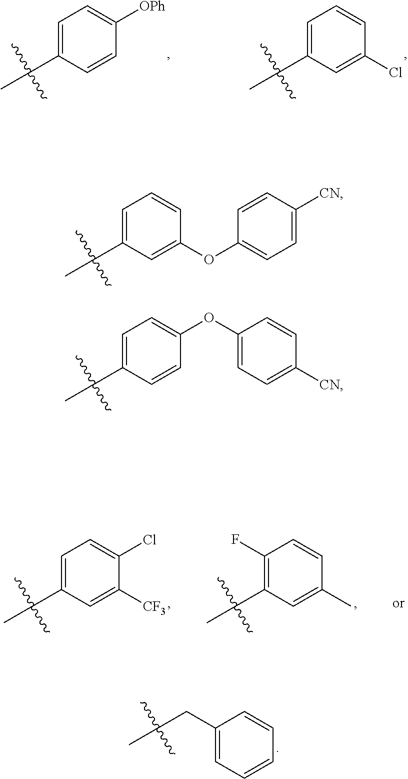

Many of the attendant features will be more readily appreciated as the same becomes better understood by reference to the following detailed description considered in connection with the accompanying drawings.

### BRIEF DESCRIPTION OF THE DRAWINGS

FIGS. 1A-1B are the western blot analysis of inhibiting EGFR phosphorylation activity by erlotinib and compound 8 in PC9 cells (a human lung adenocarcinoma), respectively.

FIG. 1C is the western blot analysis of inhibiting EGFR phosphorylation activity by erlotinib and compound 8 in H358 cells (a lung cancer cell line), respectively. D denotes DMSO.

FIG. 2 is the inhibition (%) of CIP2A expression by compounds 1-24 at a concentration of 20 μM for 24 hours in SK-Hep1 cells (hepatocellular carcinoma cell).

FIG. 3A is the western blot analysis of inhibiting EGFR phosphorylation activity by erlotinib, compound 19, and compound 22 in PC9 cells, respectively.

FIG. 3B is the cell viability (upper part) and CIP2A expressions (lower part) in response to compound 19 or erlotinib treatment in SK-Hep1 cell.

FIG. 3C is the CIP2A expressions and PARP cleavage in response to compound 19 or erlotinib treatment at a concentration of 5 μM in H358. H460, and H322 cells (lung cancer cells).

FIG. 4A is the western blot analysis of the effect of the compounds 4, 19, and 22 on phosphorylation of Akt, PARP and Actin in SK-Hep-1 cells.

FIG. 4B is flow cytometry analysis of cell death induced by compounds 4, 19, and 22 at 5 μM, after 24 h of treatment in SK-Hep-1 cells. Columns, mean (n=3); bars, SD; *P<0.05

FIG. 4C is ELISA analysis of cell death to analyze effects of compounds 4, 19, and 22 on DNA fragmentation in SK-Hep-1 cells. Columns, mean (n=3); bars, SD; *P<0.05

FIG. 5A shows the effect of CIP2A knowicjdiwn in clonogenic assay.

FIG. 5B shows the effect of okadaic acid in compound 19 induced CIP2A inhibition.

FIGS. 6A and 6B shows the tumor size changed with days of treatment by erlotinib, compound 1, and compound 9 in PLC5 xenograft tumor.

FIGS. 7A and 7B shows the tumor size changed with days of treatment by erlotinib, compound 1, and compound 9 in Huh-7 xenograft tumor.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The following abbreviations are used: CDCl₃, deuterated chloroform; DMSO-d6, dimethyl sulfoxide-d6; i-PrOH, isopropyl alcohol; EtOAc, ethyl acetate; DMF, N,N-dimethylformamide; MeOH, methanol; THF, tetrahydrofuran; EtOH, ethanol; DMSO, dimethyl sulfoxide; DIPEA, diisopropylethylamine; DCM, dichloromethane.

### Synthesis of Pyrimidine Derivatives

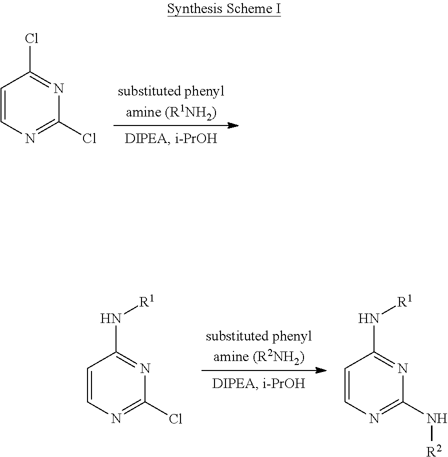

In the synthesis scheme I above, R¹ and R² can be the same or different substituted phenyl group, such as a mono-substituted phenyl group or a di-substituted phenyl group. The mono-substituted phenyl group can be

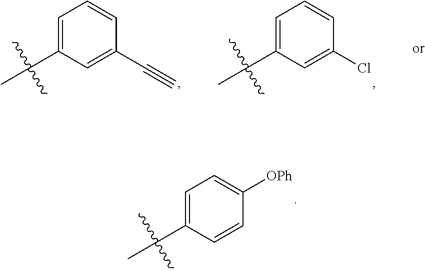

The di-substituted phenyl group can be

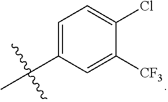 [SMILES: *c1ccc(Cl)c(C(F)(F)F)c1]

One or both of the R¹ and R² also can be a benzyl group,

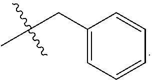 [SMILES: *Cc1ccccc1]

The general synthesis procedure of the pyrimidine derivatives is described as follow.

A solution of 2,4-dichloropyrimidine (1.0 mmol) and N,N-diisopropylethylamine (DIPEA) (100 μl) in isopropyl alcohol was added with 0.7 mmol of a substituted phenyl amine, and the mixtures was stirred in ice-bath for 30 minutes. The resulting mixture was stirred at room temperature for 8 hours. After the reaction was completed, the reaction mixture was washed with water, extracted with EtOAc, and the organic layer was dried over MgSO₄. After removal of MgSO₄ by filtration and evaporation of solvents, the crude residue was purified by chromatography on a silica gel column (silica gel columns 60, 0.063-0.200 mm or 0.040-0.063 mm, Merck; basic silica gel) using MeOH/CH₂Cl₂ as eluent (0% to 2%) to give compounds 1-7 (yield: 3-27%) below.

Embodiment 1: Synthesis of Mono-Substituted Pyrimidine Derivatives

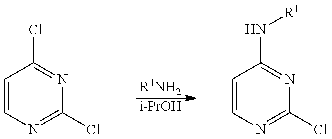

| Compound No | R¹ |
|---|---|
| 1 | 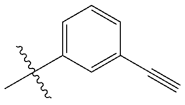 [SMILES: *c1cccc(C#C)c1] |
| 2 | 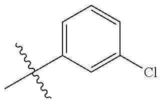 [SMILES: *c1cccc(Cl)c1] |
| 3 | 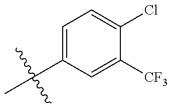 [SMILES: *c1ccc(Cl)c(C(F)(F)F)c1] |
| 4 | 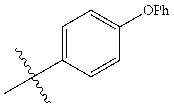 [SMILES: *c1ccc(Oc2ccccc2)cc1] |
| 5 | 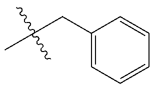 [SMILES: *Cc1ccccc1] |

The spectral data of the above compounds are listed below.

### Compound 1: 2-Chloro-N-(3-ethynylphenyl)pyrimidin-4-amine

¹H NMR (400 MHz, MeOH-d₄): δ 3.48 (s, 1H), 6.66 (d, J=6.0 Hz, 1H), 7.18 (d, J=7.6 Hz, 1H), 7.30 (t, J=7.6 Hz, 1H), 7.61 (d, J=8.4 Hz, 1H), 7.72 (s, 1H), 8.05 (d, J=6.0 Hz, 1H); ¹³C NMR (100 MHz, CDCl₃): δ 78.6, 82.8, 102.9, 123.7, 123.9, 126.5, 129.8, 130.0, 137.5, 158.5, 161.1, 162.6; HRMS calculated for C₁₂H₈ClN₃ (M+H): 230.0485. Found: 230.0478. Yield: 5%.

### Compound 2: 2-Chloro-N-(3-chlorophenyl)pyrimidin-4-amine

¹H NMR (400 MHz, CDCl₃): δ 6.59 (d, J=5.6 Hz, 1H), 7.18 (d, J=8.0 Hz, 1H), 7.22 (d, J=8.4 Hz, 1H), 7.31 (t, J=8.0 Hz, 1H), 7.36 (s, 1H), 8.15 (d, J=5.6 Hz, 1H); ¹³C NMR (100 MHz, CDCl₃): δ 102.9, 120.6, 122.6, 125.8, 130.6, 135.2, 138.4, 158.2, 160.8, 162.0; HRMS calculated for C₁₀H₇Cl₂N₃ (M+H): 240.0095. Found: 240.0101. Yield: 6%.

### Compound 3: 2-Chloro-N-(4-chloro-3-(trifluoromethyl)phenyl)pyrimidin-4-amine

¹H NMR (400 MHz, CDCl₃): δ 6.55 (d, J=5.6 Hz, 1H), 7.11 (s, 1H), 7.51 (d, J=8.4 Hz, 1H), 7.63 (d, J=8.4 Hz, 1H), 7.72 (s, 1H), 8.20 (d, J=5.6 Hz, 1H); ¹³C NMR (100 MHz, CDCl₃): δ 103.6, 120.8 (q), 123.7, 125.8, 128.1, 129.2, 129.5, 132.5, 136.2, 158.3, 160.9, 161.4; HRMS calculated for C₁₁H₆Cl₂F₃N₃ (M+H): 307.9969. Found: 307.9969. Yield: 3%.

### Compound 4: 2-Chloro-N-(4-phenoxyphenyl)pyrimidin-4-amine

¹H NMR (400 MHz, MeOH-d₄): δ 6.63 (d, J=6.0 Hz, 1H), 6.96-7.00 (m, 4H), 7.08 (t, J=7.2 Hz, 1H), 7.33 (t, J=8.0 Hz, 2H), 7.54 (d, J=8.0 Hz, 2H), 8.01 (d, J=6.0 Hz, 1H); ¹³C NMR (100 MHz, CDCl₃): δ 102.1, 119.0, 119.6, 123.7, 125.5, 129.8, 131.7, 155.6, 156.7, 158.0, 160.8, 163.0; HRMS calculated for C₁₆H₁₂ClN₃O (M−H): 296.0591. Found: 296.0583. Yield: 14%.

### Compound 5: N-Benzyl-2-chloropyrimidin-4-amine

¹H NMR (400 MHz, MeOH-d₄): δ 4.55 (s, 2H), 6.60 (d, J=5.2 Hz, 1H), 7.19-7.22 (m, 1H), 7.26-7.31 (m, 4H), 8.12 (d, J=5.2 Hz, 1H); ¹³C NMR (100 MHz, MeOH-d₄): δ 43.8, 104.4, 126.9, 127.4, 128.2, 138.3, 154.3, 160.2, 163.7; HRMS calculated for C₁₁H₁₀ClN₃ (M+H): 220.0642. Found: 220.0640. Yield: 27%.

Embodiment 2: Synthesis of Di-Substituted Pyrimidine Derivatives

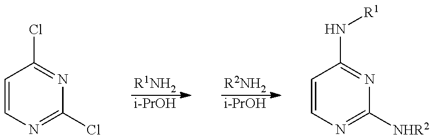

| Com - | | |
|---|---|---|
| pound | | |
| No | R¹ | R² |
| 6 | 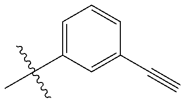 [SMILES: *c1cccc(C#C)c1] | 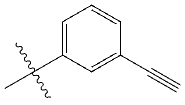 [SMILES: *c1cccc(C#C)c1] |
| 7 | 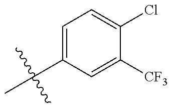 [SMILES: *c1ccc(Cl)c(C(F)(F)F)c1] | 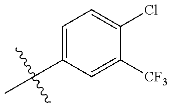 [SMILES: *c1ccc(Cl)c(C(F)(F)F)c1] |

The spectral data of the above compounds are listed below.

### Compound 6: N²,N⁴-Bis(3-ethynylphenyl)pyrimidine-2,4-diamine

¹H NMR (400 MHz, CDCl₃): δ 3.04 (s, 1H), 3.10 (s, 1H), 6.16 (d, J=6.0 Hz, 1H), 7.13 (d, J=8.0 Hz, 1H), 7.19-7.31 (m, 4H), 7.37 (d, J=8.0 Hz, 1H), 7.45 (s, 1H), 7.54 (d, J=8.4 Hz, 1H), 7.72 (s, 1H), 7.92 (brs, 1H), 8.06 (d, J=6.0 Hz, 1H); ¹³C NMR (100 MHz, CDCl₃): δ 77.0, 77.8, 83.0, 83.8, 97.2, 120.5, 122.4, 122.6, 123.0, 123.1, 125.2, 126.1, 128.0, 128.8, 129.3, 138.5, 139.7, 157.2, 159.8, 161.0; HRMS calculated for C₂₀H₁₄N₄ (M+H): 311.1297. Found: 311.1291. Yield: 10%.

### Compound 7: N²,N⁴-Bis(4-chloro-3-(trifluoromethyl)phenyl)pyrimidine-2,4-diamine

¹H NMR (400 MHz, CDCl₃): δ 6.16 (d, J=5.6 Hz, 1H), 6.60 (s, 1H), 7.10 (s, 1H), 7.38 (d, J=8.4 Hz, 1H), 7.45 (d, J=8.0 Hz, 1H), 7.60 (d, J=8.4 Hz, 1H), 7.68 (d, J=8.8 Hz, 1H), 7.72 (s, 1H), 7.92 (s, 1H), 8.12 (d, J=5.6 Hz, 1H); ¹³C NMR (100 MHz, MeOH-d₄): δ 99.6, 118.0 (q), 118.4 (q), 118.7, 118.9, 121.4, 121.6, 123.0, 123.3, 123.9, 124.1, 124.2, 124.3, 126.91-128.30 (m), 131.2, 131.4, 139.0, 139.6, 155.8, 159.1, 160.6; HRMS calculated for C₁₈H₁₀Cl₂F₆N₄ (M+H): 467.0265. Found: 467.0254. Yield: 5%.

### Synthesis of Quinazoline Derivatives

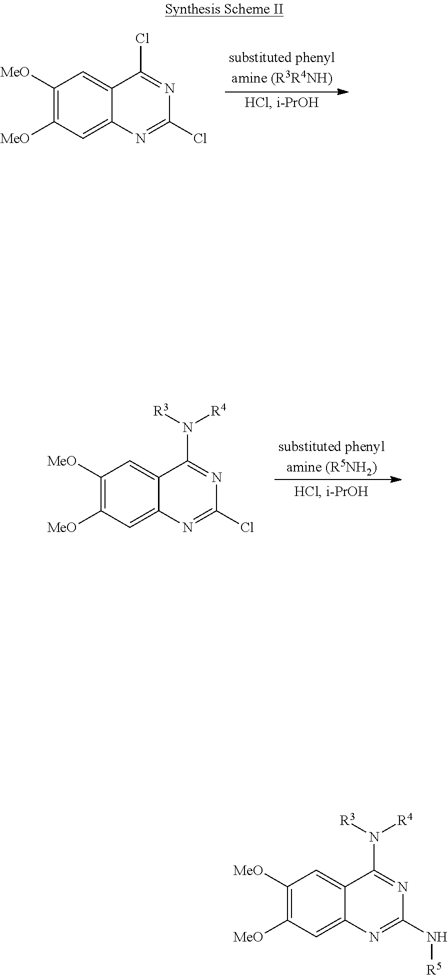

In the synthesis scheme II, R³ and R⁵ can be the same or different substituted phenyl groups, such as a mono-substituted phenyl group or a di-substituted phenyl group. The mono-substituted phenyl group can be

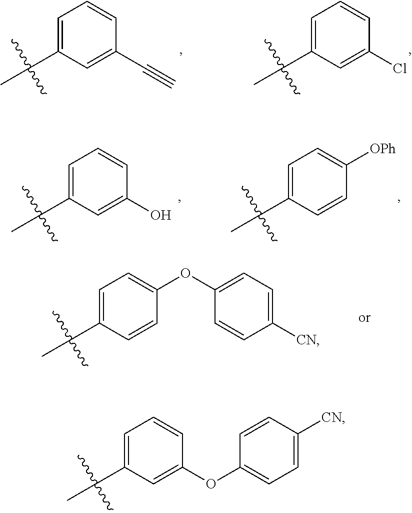

The di-substituted phenyl group can be

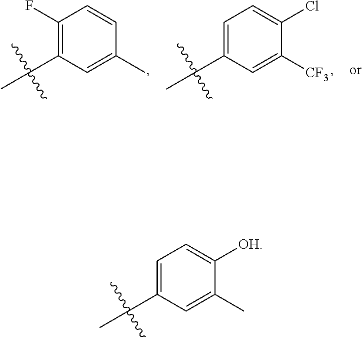

One or both of R³ and R⁵ also can be a benzyl group,

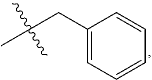 [SMILES: *Cc1ccccc1]

R⁴ can be H or methyl group.

A series of quinazoline derivatives were designed and synthesized by the general procedure illustrated in scheme II above. Based on the core quinazoline structure of these quinazoline derivatives, a commercially available dichloro-quinazoline was chosen as a starting material. A series of mono-quinazoline derivatives (compounds 8-17) were generated with various substituted phenylamines by replacement of the chloride in the quinazoline (Embodiment 3). Then, the other chloride from the mono-substitute quinazolines was replaced with various substituted phenylamines to yield compounds 18-24 (Embodiment 4).

### Embodiment 3

### Synthesis of Mono-Substituted Quinazoline Derivatives

The general synthesis procedure of the mono-substituted quinazoline derivatives are stated as follow. Substituted phenyl amine (0.8 mmol) was added to a solution of 2,4-dichloro-6,7-dimethoxyquinazoline (1.0 mmol) in isopropyl alcohol (5 ml), followed by the addition of a drop of concentrated HCl (100 μl). The resulting mixture was stirred at 60° C. for 2 hours. The mixture was filtered, and the solid was washed with isopropyl alcohol then dried under vacuum to give compounds 8, and 10-17. This procedure afforded the expected coupling product as a white or yellow solid (yield: 21%-95%).

Compound 9 was synthesized from compound 8, and the further synthesis procedure is as follow. Methyl iodide (56 μl, 0.90 mmol) was added to a solution of compound 8 (61.0 mg, 0.18 mmol) and sodium hydride (60% oil suspension, 8.63 mg, 0.36 mmol) in 2 ml of DMF cooled to 0° C. The mixture was stirred at 0° C. for 1 hour, then allowed to warm to room temperature and stirred for another 1 hour. The reaction mixture was washed with water, and then extracted with EtOAc. The organic phase was dried over MgSO₄, filtered and concentrated under reduced pressure. The crude residue was purified by chromatography on a silica gel column (silica gel columns 60, 0.063-0.200 mm or 0.040-0.063 mm, Merck; basic silica gel) using EtOAc/hexane as eluent (0% to 40%) to give compound 9.

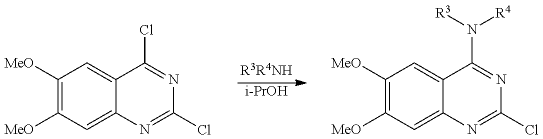

| Compound No | R³ | R⁴ |
|---|---|---|
| 8 | 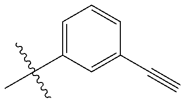 [SMILES: *c1cccc(C#C)c1] | H |
| 9 | 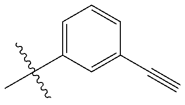 [SMILES: *c1cccc(C#C)c1] | Me |
| 10 | 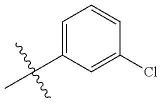 [SMILES: *c1cccc(Cl)c1] | H |
| 11 | 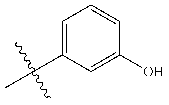 [SMILES: *c1cccc(O)c1] | H |
| 12 | 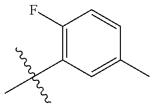 [SMILES: *c1cc(C)ccc1F] | H |
| 13 | 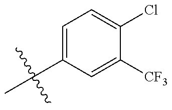 [SMILES: *c1ccc(Cl)c(C(F)(F)F)c1] | H |
| 14 | 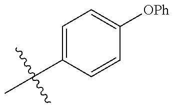 [SMILES: *c1ccc(Oc2ccccc2)cc1] | H |
| 15 | 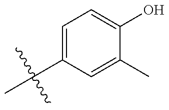 [SMILES: *c1ccc(O)c(C)c1] | H |
| 16 | 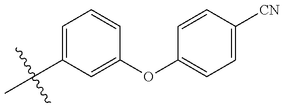 [SMILES: *c1cccc(Oc2ccc(C#N)cc2)c1] | H |
| 17 | 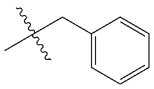 [SMILES: *Cc1ccccc1] | H |

The spectral data of the above compounds are listed below.

### Compound 8: 2-Chloro-N-(3-ethynylphenyl)-6,7-dimethoxyquinazoline-4-amine

¹H NMR (400 MHz, DMSO-d₆): δ 3.93 (s, 3H), 3.99 (s, 3H), 4.21 (s, 1H), 7.18 (s, 1H), 7.26 (d, J=8.0 Hz, 1H), 7.43 (t, J=8.0 Hz, 1H), 7.85 (d, J=8.0 Hz, 1H), 7.89 (s, 1H), 7.94 (s, 1H), 10.00 (s, 1H); ¹³C NMR (100 MHz, DMSO-d₆): δ 56.4, 57.2, 81.1, 83.8, 103.9, 106.6, 107.9, 122.2, 123.8, 126.0, 127.6, 129.2, 139.5, 148.1, 149.5, 154.1, 155.5, 158.4; HRMS calculated for C₁₈H₁₄ClN₃O₂ (M+H): 340.0853. Found: 340.0850. Yield: 94%.

### Compound 9: 2-Chloro-N-(3-ethynylphenyl)-6,7-dimethoxy-N-methyl quinazoline-4-amine

¹H NMR (400 MHz, CDCl₃): δ 3.07 (s, 1H), 3.28 (s, 3H), 3.57 (s, 3H), 3.88 (s, 3H), 6.21 (s, 1H), 7.05 (s, 1H), 7.15 (d, J=7.2 Hz, 1H), 7.31-7.37 (m, 3H); ¹³C NMR (100 MHz, CDCl₃): δ 42.2, 55.2, 56.1, 78.8, 82.0, 104.8, 106.7, 108.6, 124.2, 126.7, 129.5, 130.0, 130.2, 147.5, 147.8, 150.4, 154.4, 155.0, 161.3; HRMS calculated for C₁₉H₁₆ClN₃O₂ (M+H): 354.1009. Found: 354.1016. Yield: 60%.

### Compound 10: 2-Chloro-N-(3-chlorophenyl)-6,7-dimethoxyquinazoline-4-amine

¹H NMR (400 MHz, DMSO-d₆): δ 3.90 (s, 3H), 3.95 (s, 3H), 7.18 (s, 1H), 7.20 (d, 1H, J=8.0 Hz), 7.44 (t, J=8.0 Hz, 1H), 7.79 (d, J=8.0 Hz, 1H), 7.95 (s, 1H), 7.97 (s, 1H), 10.08 (s, 1H); ¹³C NMR (100 MHz, DMSO-d₆): δ 56.5, 57.1, 103.3, 106.6, 107.8, 121.3, 122.4, 124.1, 130.5, 133.1, 140.7, 148.2, 149.6, 154.1, 155.6, 158.2; HRMS calculated for C₁₆H₁₃Cl₂N₃O₂ (M+H): 350.0463. Found: 350.0466. Yield: 75%.

### Compound 11: 3-(2-Chloro-6,7-dimethoxyquinazoline-4-ylamino) phenol

¹H NMR (400 MHz, DMSO-d₆): δ 3.15 (s, 1H), 3.91 (s, 3H), 3.94 (s, 3H), 6.59 (d, J=6.8 Hz, 1H), 7.13-7.21 (m, 4H), 7.98 (s, 1H), 9.96 (s, 1H); ¹³C NMR (100 MHz, DMSO-d₆): δ 56.4, 56.9, 58.5, 103.3, 106.4, 107.7, 110.6, 112.1, 114.2, 129.5, 139.8, 147.6, 149.5, 154.2, 155.4, 158.0; HRMS calculated for C₁₆H₁₄ClN₃O₃ (M+H): 332.0802. Found: 332.0810. Yield: 60%.

### Compound 12: 2-Chloro-N-(2-fluoro-5-methylphenyl)-6,7-dimethoxy quinazoline-4-amine

¹H NMR (400 MHz, DMSO-d₆): δ 2.32 (s, 3H), 3.87 (s, 3H), 3.92 (s, 3H), 7.12-7.16 (m, 2H), 7.22 (d, J=10.4 Hz, 1H), 7.29 (d, J=7.6 Hz, 1H), 7.90 (s, 1H), 10.05 (s, 1H); ¹³C NMR (100 MHz, DMSO-d₆): δ 20.1, 56.0, 56.3, 102.8, 105.7, 106.8, 115.61, 115.8, 124.9, 125.0, 128.1, 128.2, 128.5, 133.6, 133.7, 146.9, 149.1, 153.9, 153.9, 155.1, 156.3, 159.1; HRMS calculated for C₁₇H₁₆ClFN₃O₂ (M+H): 348.0915. Found: 348.0911. Yield: 60%.

### Compound 13: 2-Chloro-N-(4-chloro-3-(trifluoromethyl)phenyl)-6,7-dimethoxyquinazoline-4-amine

¹H NMR (400 MHz, DMSO-d₆): δ 3.92 (s, 3H), 3.96 (s, 3H), 7.19 (s, 1H), 7.75 (d, J=8.8 Hz, 1H), 7.99 (s, 1H), 8.19 (d, J=8.8 Hz, 1H), 8.42 (s, 1H), 10.32 (s, 1H); ¹³C NMR (100 MHz, DMSO-d₆): δ 56.0, 56.8, 102.8, 105.9, 107.3, 118.7, 120.9, 120.9, 121.0, 121.0, 121.5, 124.2, 124.5, 125.9, 126.2, 126.5, 126.9, 131.6, 138.3, 147.5, 149.2, 153.1, 155.2, 157.2; HRMS calculated for C₁₇H₁₂Cl₂F₃N₃O₂ (M+H): 418.0337. Found: 418.0340. Yield: 86%.

### Compound 14: 2-Chloro-6,7-dimethoxy-N-(4-phenoxyphenyl) quinazoline-4-amine

¹H NMR (400 MHz, DMSO-d₆): δ 3.91 (s, 3H), 3.94 (s, 3H), 7.03 (d, J=8.6 Hz, 2H), 7.07 (d, J=8.8 Hz, 2H), 7.11-7.16 (m, 2H), 7.40 (t, J=6.0 Hz, 2H), 7.71 (d, J=8.8 Hz, 2H), 7.90 (s, 1H), 9.93 (s, 1H); ¹³C NMR (100 MHz, DMSO-d₆): δ 56.5, 57.0, 103.4, 105.9, 107.5, 118.8, 119.5, 123.8, 125.3, 130.5, 134.3, 146.9, 149.5, 153.6, 153.9, 155.5, 157.4, 158.4; HRMS calculated for C₂₂H₁₈ClN₃O₃ (M+H): 408.1115. Found: 408.1121. Yield: 55%.

### Compound 15: 4-(2-Chloro-6,7-dimethoxyquinazoline-4-ylamino)-3-methylphenol

¹H NMR (400 MHz, DMSO-d₆): δ 2.70 (s, 3H), 3.89 (s, 3H), 3.90 (s, 3H), 6.64 (d, J=8.4 Hz, 1H), 6.71 (s, 1H), 7.04 (d, J=8.4 Hz, 1H), 7.11 (s, 1H), 7.83 (s, 1H), 9.67 (s, 1H); ¹³C NMR (100 MHz, DMSO-d₆): δ 18.5, 56.4, 56.5, 102.8, 107.0, 107.3, 113.5, 117.3, 128.0, 129.2, 136.7, 148.1, 149.2, 155.1, 155.5, 156.5, 160.1; HRMS calculated for C₁₇H₁6 ClN₃O₃ (M+H): 346.0958. Found: 346.0951. Yield: 23%.

### Compound 16: 4-(3-(2-Chloro-6,7-dimethoxyquinazoline-4-ylamino) phenoxy)benzonitrile

¹H NMR (400 MHz, DMSO-d₆): δ 3.91 (s, 3H), 3.94 (s, 3H), 6.94 (d, J=8.0 Hz, 1H), 7.17 (s, 1H), 7.21 (d, J=8.8 Hz, 2H), 7.50 (t, J=8.0 Hz, 1H), 7.65 (m, 2H), 7.86 (d, J=8.8 Hz, 2H), 7.90 (s, 1H), 10.00 (s, 1H); ¹³C NMR (100 MHz, DMSO-d₆): δ 56.0, 56.2, 102.1, 105.3, 106.6, 107.2, 113.8, 115.4, 118.4, 118.6, 118.7, 130.2, 134.6, 140.4, 148.2, 149.0, 153.9, 154.6, 155.0, 157.6, 160.7; HRMS calculated for C₂₃H₁₇ClN₄O₃ (M−H): 431.0911. Found: 431.0909. Yield: 74%.

### Compound 17: N-Benzyl-2-chloro-6,7-dimethoxyquinazoline-4-amine

¹H NMR (400 MHz, MeOH-d₄): δ 3.90 (s, 3H), 3.92 (s, 3H), 4.79 (s, 2H), 6.96 (s, 1H), 7.23 (t, J=7.2 Hz, 1H), 7.31 (t, J=7.2 Hz, 2H), 7.39 (d, J=7.2 Hz, 2H), 7.47 (s, 1H); ¹³C NMR (100 MHz, DMSO-d₆): δ 43.4, 55.8, 56.0, 102.2, 106.5, 106.8, 126.9, 127.4, 128.3, 138.9, 147.2, 148.5, 154.4, 155.0, 159.9; HRMS calculated for C₁₇H₁₆ClN₃O₂ (M+H): 330.1009. Found: 330.1007. Yield: 21%.

### Embodiment 4

### Synthesis of Di-Substituted Quinazoline Derivatives

The general synthesis procedure of the di-substituted quinazoline derivatives are stated as follow. Substituted phenyl amine (0.5 mmol) were added to a solution of compound 8 (0.2 mmol) in isopropyl alcohol (3 ml), followed by the addition of a drop of concentrated HCl (100 μl). The resulting solution was heated by using microwave irradiation to 150° C. for 30 min. After cooling, the mixture was filtered, and the solid was washed with isopropyl alcohol. The crude solid was dissolved in CH₂Cl₂ and washed with saturated NaHCO₃ solution. The organic phase was dried over MgSO₄, filtered and concentrated under reduced pressure. The crude residue was purified by chromatography on a silica gel column (silica gel columns 60, 0.063-0.200 mm or 0.040-0.063 mm, Merck; basic silica gel) using MeOH/CH₂Cl₂ as eluent (0% to 5%) to give compounds 18-24 (yield: 20-80%).

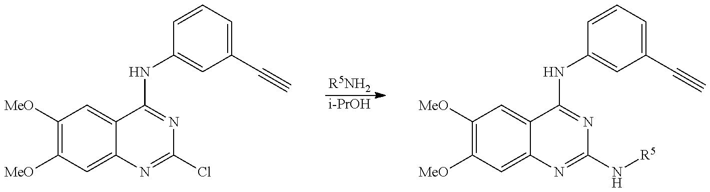

| Compound | |
|---|---|
| No | R⁵ |
| 18 | 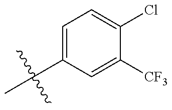 [SMILES: *c1ccc(Cl)c(C(F)(F)F)c1] |
| 19 | 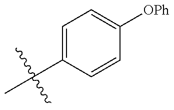 [SMILES: *c1ccc(Oc2ccccc2)cc1] |
| 20 | 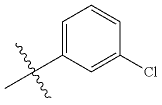 [SMILES: *c1cccc(Cl)c1] |
| 21 | 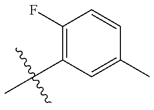 [SMILES: *c1cc(C)ccc1F] |
| 22 | 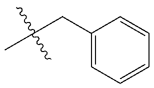 [SMILES: *Cc1ccccc1] |
| 23 | 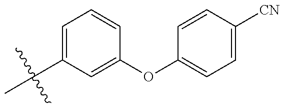 [SMILES: *c1cccc(Oc2ccc(C#N)cc2)c1] |
| 24 | 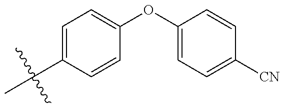 [SMILES: *c1ccc(Oc2ccc(C#N)cc2)cc1] |
| 25 | 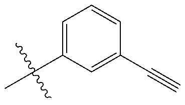 [SMILES: *c1cccc(C#C)c1] |
| 26 | 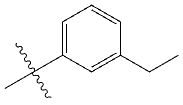 [SMILES: *c1cccc(CC)c1] |
| 27 | 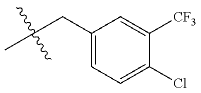 [SMILES: *Cc1ccc(Cl)c(C(F)(F)F)c1] |
| 28 | 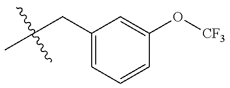 [SMILES: *Cc1cccc(OC(F)(F)F)c1] |
| 29 | 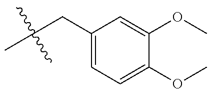 [SMILES: *Cc1ccc(OC)c(OC)c1] |
| 30 | 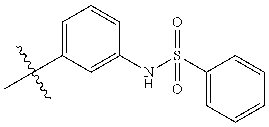 [SMILES: *c1cccc(NS(=O)(=O)c2ccccc2)c1] |
| 31 | 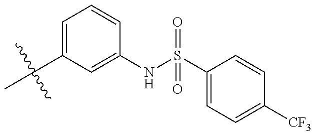 [SMILES: *c1cccc(NS(=O)(=O)c2ccc(C(F)(F)F)cc2)c1] |
| 32 | 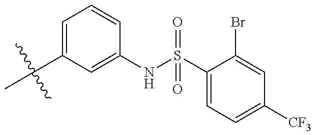 [SMILES: *c1cccc(NS(=O)(=O)c2ccc(C(F)(F)F)cc2Br)c1] |
| 33 | 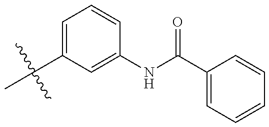 [SMILES: *c1cccc(NC(=O)c2ccccc2)c1] |

The spectral data of the above compounds are listed below.

### Compound 18: N²-(4-Chloro-3-(trifluoromethyl)phenyl)-N⁴-(3-ethynylphenyl)-6,7-dimethoxy-quinazoline-2,4-diamine

¹H NMR (400 MHz, MeOH-d₄): δ 3.47 (s, 1H), 3.91 (s, 3H), 3.93 (s, 3H), 6.88 (s, 1H), 7.22 (d, J=7.6 Hz, 2H), 7.31 (t, J=8.0 Hz, 1H), 7.34 (d, J=9.2 Hz, 1H), 7.53 (s, 1H), 7.80 (d, J=8.0 Hz, 1H), 7.83 (s, 1H), 8.01-8.04 (m, 2H); ¹³C NMR (100 MHz, MeOH-d₄): δ 54.9, 55.4, 77.1, 77.2, 83.1, 101.9, 105.0, 105.1, 117.1-117.2 (q), 121.5, 121.7, 121.9 (d), 122.5, 122.7, 122.9, 123.3, to 124.4, 125.5, 126.9, 127.3, 127.6, 128.3, 128.5, 131.17, 139.5, 140.3, 147.0, 148.3, 155.1, 155.5, 157.8; HRMS calculated for C₂₅H₁₈ClF₃N₄O₂ (M+H): 499.1149. Found: 499.1142. Yield: 33%.

### Compound 19: N⁴-(3-Ethynylphenyl)-6,7-dimethoxy-N²-(4-phenoxyphenyl) quinazoline-2,4-diamine

¹H NMR (400 MHz, DMSO-d₆): δ 3.93 (s, 3H), 3.94 (s, 3H), 4.18 (s, 1H), 6.98 (d, 4H, J=8.8 Hz), 7.13 (s, 1H), 7.15 (d, 1H, J=7.6 Hz), 7.33-7.45 (m, 6H), 7.69 (d, 1H, J=7.6 Hz), 7.75 (s, 1H), 8.10 (s, 1H), 10.33 (s, 1H), 10.90 (s, 1H); ¹³C NMR (100 MHz, CDCl₃): δ 56.1, 56.2, 77.6, 83.2, 100.0, 104.4, 106.3, 117.9, 120.0, 120.8, 122.4, 122.5, 122.7, 125.2, 127.7, 128.8, 129.5, 135.9, 138.6, 146.7, 149.2, 151.3, 155.1, 155.9, 156.9, 158.2; HRMS calculated for C₃₀H₂₄N₄O₃ (M+H): 489.1927. Found: 489.1925. Yield: 40%.

### Compound 20: N²-(3-Chlorophenyl)-N⁴-(3-ethynylphenyl)-6,7-dimethoxy quinazoline-2,4-diamine

¹H NMR (400 MHz, MeOH-d₄): δ 3.47 (s, 1H), 3.93 (s, 3H), 3.94 (s, 3H), 6.88 (d, J=8.0 Hz, 1H), 6.92 (s, 1H), 7.17 (t, J=8.0 Hz, 1H), 7.22 (d, J=7.6 Hz, 1H), 7.34 (t, J=8.0 Hz, 1H), 7.53 (d, J=8.4 Hz, 1H), 7.58 (s, 1H), 7.80 (s, 2H), 7.87 (d, J=8.4 Hz, 1H); ¹³C NMR (100 MHz, MeOH-d₄): δ 54.9, 55.4, 77.1, 83.1, 102.0, 104.9, 104.9, 117.0, 118.4, 120.5, 122.6, 123.0, 125.5, 126.9, 128.5, 129.3, 133.7, 139.6, 142.3, 146.9, 148.5, 155.1, 155.8, 157.9; HRMS calculated for C₂₄H₁₉ClN₄O₂ (M+H): 431.1275. Found: 431.1280. Yield: 34%.

### Compound 21: N⁴-(3-Ethynylphenyl)-N²-(2-fluoro-5-methylphenyl)-6,7-dimethoxyquinazoline-2,4-diamine

¹H NMR (400 MHz, MeOH-d₄): δ 2.18 (s, 3H), 3.45 (s, 1H), 3.95 (s, 3H), 3.96 (s, 3H), 6.75 (brs, 1H), 6.91-6.98 (m, 2H), 7.23 (d, J=7.6 Hz, 1H), 7.31 (t, J=8.0 Hz, 1H), 7.62 (s, 1H), 7.75-7.77 (m, 2H), 7.91 (d, J=7.6 Hz, 1H); ¹³C NMR (100 MHz, CDCl₃): δ 21.2, 56.1, 56.1, 75.5, 100.3, 104.4, 105.5, 114.0, 114.2, 121.4, 122.2, 122.3, 122.7, 122.7, 125.3, 127.6, 127.7, 127.8, 128.9, 133.7, 133.7, 138.6, 146.9, 148.1, 149.7, 152.1, 155.2, 155.2, 157.1; HRMS calculated for C₂₅H₂₁FN₄O₂ (M+H): 429.1727. Found: 429.1721. Yield: 20%.

### Compound 22: N²-Benzyl-N⁴-(3-ethynylphenyl)-6,7-dimethoxy quinazoline-2,4-diamine

¹H NMR (400 MHz, DMSO-d₆): δ 3.84 (s, 3H), 3.85 (s, 3H), 4.15 (s, 1H), 4.53 (d, J=6.4 Hz, 2H), 6.75 (s, 1H), 7.10-7.19 (m, 3H), 7.27 (t, J=8.0 Hz, 1H), 7.28 (d, J=7.2 Hz, 2H), 7.33 (d, J=7.2 Hz, 2H), 7.63 (s, 1H), 7.90 (s, 1H), 9.14 (s, 1H); ¹³C NMR (100 MHz, CDCl₃): δ 29.3, 45.2, 55.7, 55.9, 76.8, 83.0, 100.1, 103.4, 105.2, 121.6, 122.2, 124.2, 126.7, 126.9, 127.1, 128.1, 128.4, 138.5, 139.3, 145.7, 154.7, 156.5, 158.0; HRMS calculated for C₂₅H₂₂N₄O₂ (M+H): 411.1821. Found: 411.1826. Yield: 15%.

### Compound 23: 4-(3-(4-(3-Ethynylphenylamino)-6,7-dimethoxyquinazolin-2-ylamino)phenoxy)benzonitrile

¹H NMR (400 MHz, CDCl₃): δ 3.05 (s, 1H), 3.93 (s, 3H), 3.95 (s, 3H), 6.61 (d, J=7.6 Hz, 1H), 6.92 (s, 1H), 6.94 (s, 1H), 7.01 (d, J=9.2 Hz, 2H), 7.20 (d, J=7.6 Hz, 1H), 7.26 (t, J=8.0 Hz, 2H), 7.35 (d, J=7.2 Hz, 2H), 7.54 (d, J=9.2 Hz, 2H), 7.64 (s, 1H), 7.66 (d, J=8.0 Hz, 1H), 7.73 (s, 1H); ¹³C NMR (100 MHz, CDCl₃): δ 56.1, 56.2, 77.6, 83.2, 100.1, 104.6, 105.3, 106.3, 110.5, 112.8, 115.2, 118.0, 119.0, 122.7, 122.8, 125.4, 127.9, 128.8, 130.1, 133.9, 138.5, 142.1, 147.0, 148.9, 155.1, 155.1, 155.3, 157.0, 161.6; HRMS calculated for C₃₁H₂₃N₅O₃ (M+H): 514.1879. Found: 514.1888. Yield: 80%.

### Compound 24: 4-(4-(4-(3-Ethynylphenylamino)-6,7-dimethoxyquinazolin-2-ylamino)phenoxy)benzonitrile

¹H NMR (400 MHz, DMSO-d₆): δ 3.92 (s, 3H), 3.94 (s, 3H), 4.22 (s, 1H), 7.06 (d, J=8.4 Hz, 2H), 7.10-7.15 (m, 3H), 7.36 (d, J=7.6 Hz, 1H), 7.43 (t, J=8.0 Hz, 1H), 7.53 (d, J=7.6 Hz, 2H), 7.68 (d, J=7.6 Hz, 1H), 7.74 (s, 1H), 7.85 (d, J=8.4 Hz, 2H), 8.03 (s, 1H); ¹³C NMR (100 MHz, CDCl₃): δ 56.1, 56.2, 77.6, 83.2, 100.2, 104.6, 105.0, 106.3, 117.3, 119.0, 120.6, 121.0, 122.6, 122.7, 125.5, 127.8, 128.8, 134.0, 137.6, 138.6, 146.8, 148.6, 149.2, 155.1, 155.7, 157.0, 162.4; HRMS calculated for C₃₁H₂₃N₅O₃ (M+H): 514.1879. Found: 514.1876. Yield: 75%.

### Compound 25: N²,N⁴-Bis(3-ethynylphenyl)-6,7-dimethoxyquinazoline-2,4-diamine

¹H NMR (400 MHz, DMSO-d₆) δ 3.92 (s, 3H, OCH₃), 3.94 (s, 3H, OCH₃), 4.22 (s, 1H), 4.24 (s, 1H), 7.13 (s, 1H, ArH), 7.25 (d, J=8.0 Hz, 1H, ArH), 7.32-7.36 (m, 2H, ArH), 7.44 (t, J=8.0 Hz, 1H, ArH), 7.53 (s, 2H, ArH), 7.69 (s, 1H, ArH), 7.73 (d, J=8.0 Hz, 1H, ArH), 8.05 (s, 1H, ArH); ¹³C NMR (100 MHz, CDCl₃) δ 56.0, 56.1, 77.0, 77.54, 83.3, 83.9, 100.1, 104.5, 106.2, 119.7, 122.2, 122.2, 122.5, 122.6, 125.0, 125.4, 127.6, 128.7, 128.9, 138.6, 140.1, 146.7, 148.9, 154.9, 155.4, 156.8; HRMS calculated for C₂₆H₂₀N₄O₂ [M⁺+H] 421.1665. found 421.1671.

### Compound 26: N²-(3-ethylphenyl)-N⁴-(3-ethynylphenyl)-6,7-dimethoxy quinazoline-2,4-diamine

¹H NMR (400 MHz, DMSO-d₆) δ 1.09 (t, J=7.6 Hz, 3H, CH₃), 2.52 (q, J=7.6 Hz, 2H, CH₂), 3.92 (s, 3H, OCH₃), 3.93 (s, 3H, OCH₃), 4.23 (s, 1H), 7.00 (d, J=6.8 Hz, 1H, ArH), 7.11 (s, 1H, ArH), 7.23-7.27 (m, 3H, ArH), 7.37-7.43 (m, 2H, ArH), 7.71 (d, J=8.0 Hz, 1H, ArH), 7.74 (s, 1H, ArH), 8.06 (s, 1H, ArH); ¹³C NMR (100 MHz, DMSO-d₆) δ 15.3, 28.0, 56.2, 56.3, 81.1, 82.8, 98.5, 102.9, 105.0, 119.1, 120.8, 122.1, 124.0, 125.5, 127.8, 128.7, 128.9, 129.1, 135.8, 136.6, 137.4, 144.6, 147.2, 150.6, 155.9, 158.5; HRMS calculated for C₂₆H₂₄N₄O₂ [M⁺+H] 425.1978. found 425.1978.

### Compound 27: N²-(4-Chloro-3-(trifluoromethyl)benzyl)-N⁴-(3-ethynyl phenyl)-6,7-dimethoxyquinazoline-2,4-diamine

¹H NMR (400 MHz, MeOH-d₄) δ 3.43 (s, 1H), 3.91 (s, 3H, OCH₃), 3.92 (s, 3H, OCH₃), 4.62 (s, 2H, CH₂), 6.84 (s, 1H, ArH), 7.17 (d, J=8.0 Hz, 1H, ArH), 7.22 (t, J=8.0 Hz, 1H), 7.45 (d, J=8.0 Hz, 1H, ArH), 7.50 (d, J=7.6 Hz, 1H, ArH), 7.56 (s, 1H, ArH), 7.62 (d, J=8.0 Hz, 1H, ArH), 7.70 (s, 1H, ArH), 7.86 (s, 1H, ArH); ¹³C NMR (100 MHz, CDCl₃) δ 44.5, 56.0, 56.2, 78.2, 83.2, 100.3, 103.9, 105.6, 121.4, 121.9, 122.6, 124.1, 124.9, 126.44-126.48 (t), 127.5, 127.6, 127.9, 128.6, 130.4, 131.3, 131.6, 138.6, 139.3, 146.3, 155.1, 157.0, 158.1; HRMS calculated for C₂₆H₂₀ClF₃N₄O₂ [M⁺+H] 513.1305. found 513.1309.

### Compound 28: N⁴-(3-Ethynylphenyl)-6,7-dimethoxy-N²-(3-(trifluoro methoxy)benzyl)quinazoline-2,4-diamine

¹H NMR (400 MHz, MeOH-d₄) δ 3.42 (s, 1H), 3.88 (s, 3H, OCH₃), 3.90 (s, 3H, OCH₃), 4.61 (s, 2H, CH₂), 6.81 (s, 1H, ArH), 7.05 (d, J=7.6 Hz, 1H, ArH), 7.14 (d, J=7.6 Hz, 1H, ArH), 7.17-7.21 (m, 2H, ArH), 7.30 (s, 1H, ArH), 7.32 (t, J=7.6 Hz, 1H, ArH), 7.51 (s, 1H, ArH), 7.66 (d, J=7.6 Hz, 1H, ArH), 7.87 (s, 1H, ArH); ¹³C NMR (100 MHz, MeOH-d₄) δ 45.3, 56.2, 56.8, 78.1, 84.3, 103.6, 105.1, 105.3, 119.8, 120.4, 120.4, 122.9, 123.6, 123.7, 126.5, 126.6, 127.9, 129.3, 130.6, 140.7, 144.6, 147.2, 149.9, 150.4, 150.4, 156.2, 159.1, 159.8; HRMS calculated for C₂₆H₂₁F₃N₄O₃ [M⁺+H] 549.1644. found 549.1639.

### Compound 29: N²-(3,4-dimethoxybenzyl)-N⁴-(3-ethynylphenyl)-6,7-dimethoxyquinazoline-2,4-diamine

¹H NMR (400 MHz, MeOH-d₄) δ 3.45 (s, 1H), 3.69 (s, 3H, OCH₃), 3.75 (s, 3H, OCH₃), 3.90 (s, 3H, OCH₃), 3.93 (s, 3H, OCH₃), 4.52 (s, 2H, CH₂), 6.83-6.88 (m, 3H, ArH), 6.95 (s, 1H, ArH), 7.17 (dt, J=8.0 Hz, 1H, ArH), 7.25 (t, J=8.0 Hz, 1H, ArH), 7.56 (s, 1H, ArH), 7.74 (d, 1H, ArH), 7.96 (s, 1H, ArH); ¹³C NMR (100 MHz, CDCl₃) δ 14.1, 29.6, 44.7, 55.8, 56.2, 61.6, 77.3, 83.0, 102.4, 102.8, 110.7, 111.0, 119.4, 122.6, 122.9, 125.1, 128.3, 128.7, 130.8, 138.2, 146.7, 148.2, 149.0, 155.7, 157.6, 165.2, 165.7; HRMS calculated for C₂₇H₂₆N₄O₄ [M⁺+H] 471.2032. found 471.2031.

### Compound 30: N-(3-(4-(3-ethynylphenylamino)-6,7-dimethoxyquinazolin-2-ylamino)phenyl)benzenesulfonamide

¹H NMR (400 MHz, MeOH-d₄) δ 3.47 (s, 1H), 3.77 (s, 3H), 3.86 (s, 3H), 6.66 (d, J=8.0 Hz, 1H), 6.82 (s, 1H), 7.04 (t, J=8.0 Hz, 1H), 7.19 (d, J=8.0 Hz, 1H), 7.26 (t, J=8.0 Hz, 1H), 7.30 (d, J=8.0 Hz, 1H), 7.40 (t, J=8.0 Hz, 3H), 7.46-7.48 (m, 2H), 7.53 (s, 1H), 7.75 (m, 4H); ¹³C NMR (100 MHz, MeOH-d₄) δ 56.3, 56.8, 78.8, 84.4, 103.4, 104.7, 105.7, 113.4, 115.6, 117.5, 123.9, 124.6, 127.0, 128.2, 128.7, 129.8, 129.9, 130.3, 133.8, 139.3, 140.4, 141.0, 141.8, 146.5, 148.3, 155.7, 156.6, 159.13; HRMS calcd for C₃₀H₂₅N₅O₄S [M⁺+H] 552.1627. found 552.1707. Yield: 45%.

### Compound 31: N-(3-(4-(3-Ethynylphenylamino)-6,7-dimethoxyquinazolin-2-ylamino)phenyl)-3-(trifluoromethyl)benzenesulfonamide

¹H NMR (400 MHz, DMSO-d₆) δ 3.94 (s, 6H), 4.23 (s, 1H), 6.88 (d, ¹H NMR (400 MHz, MeOH-d₄) 8, 1H), 7.13 (s, 2H), 7.19 (t, J=8.0 Hz, 1H), 7.35-7.43 (m, 3H), 7.70-7.73 (m, 2H), 7.78 (t, J=8.0 Hz, 1H), 8.01 (d, J=8.0 Hz, 1H), 8.05-8.07 (m, 2H), 8.11 (s, 1H); ¹³C NMR (100 MHz, DMSO-d₆) δ 56.7, 57.1, 81.6, 83.3, 99.3, 103.6, 105.4, 114.2, 115.0, 116.8, 118.9, 122.3, 122.4, 122.8, 123.6, 124.0, 125.1, 125.8, 126.3, 127.8, 128.0, 129.3, 129.8, 129.9, to 130.1, 130.4, 130.8, 131.2, 131.4, 136.8, 137.9, 138.2, 141.0, 147.8, 151.1, 156.3, 158.8, 158.9; HRMS calcd for C₃₁H₂₄F₃N₅O₄S [M⁺+H] 620.1501. found 620.1546. Yield: 30%.

### Compound 32: 2-Bromo-N-(3-(4-(3-ethynylphenylamino)-6,7-dimethoxy quinazolin-2-ylamino)phenyl)-4-(trifluoromethyl)benzenesulfonamide

¹H NMR (400 MHz, MeOH-d₄) δ 3.46 (s, 1H), 3.94 (s, 6H), 6.73 (d, J=8.0 Hz, 1H), 7.00 (s, 1H), 7.06 (t, J=8.0 Hz, 1H), 7.20 (d, J=8.0 Hz, 1H), 7.29-7.35 (m, 2H), 7.59 (s, 1H), 7.69 (d, J=8.4 Hz, 1H), 7.74 (s, 1H), 7.80 (s, 1H), 7.87 (d J=8.4 Hz, 1H), 8.01 (s, 1H), 8.26 (d, J=8.4 Hz, 1H); ¹³C NMR (100 MHz, MeOH-d₄) δ 56.4, 56.8, 78.6, 84.4, 103.4, 106.3, 106.4, 112.6, 114.6, 117.0, 121.8, 122.5, 123.9, 124.3, 125.2, 125.6, 125.7, 126.7, 128.2, 129.8, 130.2, 133.2, 133.3, 134.3, 135.9, 136.2, 138.0, 141.0, 143.1, 143.7, 148.2, 149.6, 156.4, 157.1, 159.2; HRMS calcd for C₃₁H₂₃BrF₃N₅O₄S [M⁺+H] 698.0606. found 698.0682. Yield: 40%.

### Compound 33: N-(3-(4-(3-ethynylphenylamino)-6,7-dimethoxy quinazolin-2-ylamino)phenyl)benzamide

¹H NMR (400 MHz, DMSO-d₆) δ 3.49 (s, 6H), 4.24 (s, 1H), 7.14 (s, 1H), 7.29-7.39 (m, 3H), 7.51-7.62 (m, 5H), 7.79 (s, 2H), 7.88 (s, 1H), 7.94 (d, J 8.0 Hz, 2H), 8.13 (s, 1H); ¹³C NMR (100 MHz, MeOH-d₄) δ 55.7, 56.1, 80.6, 82.4, 98.3, 102.5, 104.4, 113.7, 116.5, 117.4, 121.4, 124.8, 127.0, 127.1 (2C), 127.8 (2C), 128.3, 128.5, 131.1, 134.2, 135.5, 136.2, 136.9, 139.2, 139.2, 146.7, 150.3, 155.4, 157.8, 165.0; HRMS calcd for C₃₁H₂₆N₅O₃ [M⁺+H] 516.1957. found 516.2025. Yield: 70%.

### Embodiment 5

### Synthesis of Di-Substituted Quinazoline Derivatives

The R⁴ and R⁵ listed in the Table below can be paired arbitrarily.

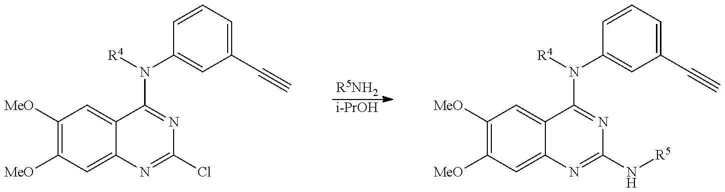

| R⁴ | R⁵ |
|---|---|
| 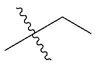 [SMILES: *CC] | 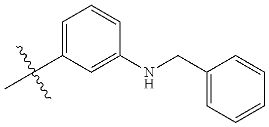 [SMILES: *c1cccc(NCc2ccccc2)c1] |
| 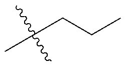 [SMILES: *CCC] | 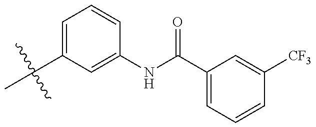 [SMILES: *c1cccc(NC(=O)c2cccc(C(F)(F)F)c2)c1] |
| 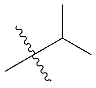 [SMILES: *C(C)C] | 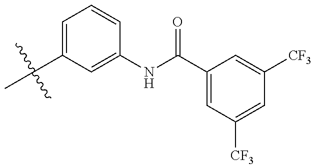 [SMILES: *c1cccc(NC(=O)c2cc(C(F)(F)F)cc(C(F)(F)F)c2)c1] |
| 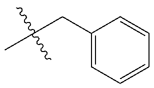 [SMILES: *Cc1ccccc1] | 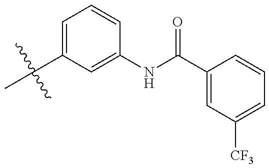 [SMILES: *c1cccc(NC(=O)c2cccc(C(F)(F)F)c2)c1] |
| 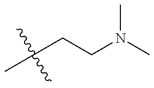 [SMILES: *CCN(C)C] | 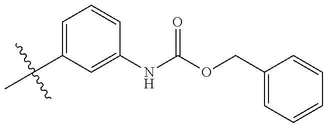 [SMILES: *c1cccc(NC(=O)OCc2ccccc2)c1] |
| 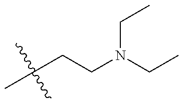 [SMILES: *CCN(CC)CC] | 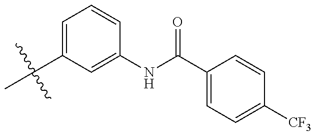 [SMILES: *c1cccc(NC(=O)c2ccc(C(F)(F)F)cc2)c1] |
| 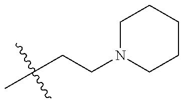 [SMILES: *CCN1CCCCC1] | 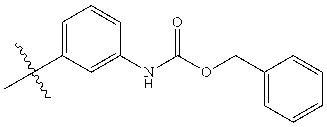 [SMILES: *c1cccc(NC(=O)OCc2ccccc2)c1] |
| | 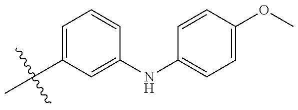 [SMILES: *c1cccc(Nc2ccc(OC)cc2)c1] |

### Pharmaceutical Composition and Medical Application Thereof

In one aspect, the present invention directs to a pharmaceutical composition. The pharmaceutical composition comprises an effective amount of a compound having a chemical structure (I), (II), (III), or (VII) below and a pharmaceutically acceptable carrier.

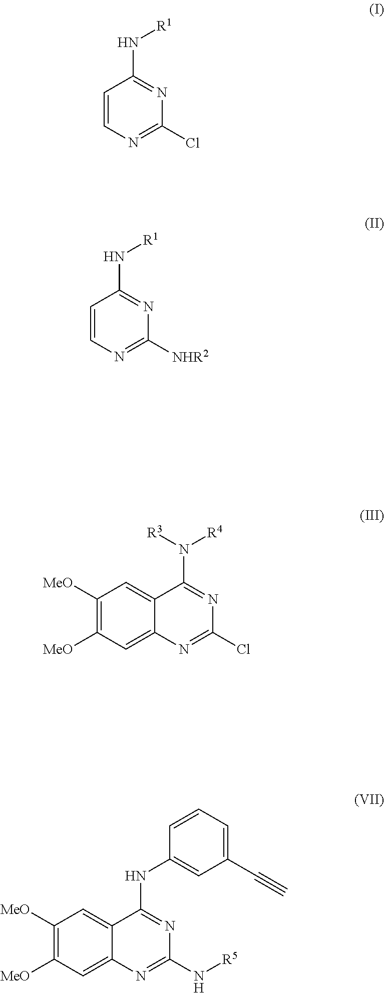

The R³ above is

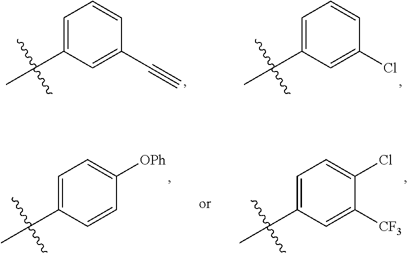

and R⁴ is H or methyl group. The R⁵ above is

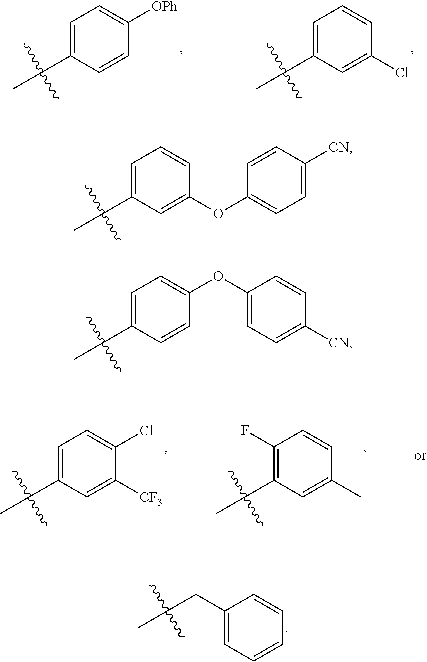

In another aspect, the present invention directs to a method of inhibiting the expression of cancerous inhibitor of PP2A. The method comprises contacting a cell with an effective amount of a compound having the chemical structure (I), (II), (III), or (VII) above.

In yet another aspect, the present invention directs to a method of treating cancer. The method comprises administrating an effective amount of a compound having a chemical structure (I), (II), (III), or (VII) above by a needed subject. The cancer above can be a hepatocellular carcinoma or a lung cancer.

### Inhibition of EGFR Kinase Activation

Quinazolines have been used as a scaffold for synthesizing a variety of pharmacological compounds. For example, antagonists of human adenosine A3 receptor, inhibitors of histone lysine methyltransferase G9a, inhibitors of poly(ADP-ribose)polymerase, an inhibitor of protein kinase c isotypes, agonists of histamine H4 receptor and inhibitors of thymidylate synthase inhibitors. Some quinazoline derivatives having amino substitutes at position 4 of the quinazoline structure have been demonstrated to be inhibitors of epidermal growth factor receptor (EGFR) kinase. EGFR kinase is a receptor tyrosine kinase, which regulates cell proliferation. The numbering of quinazoline is shown below.

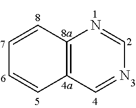

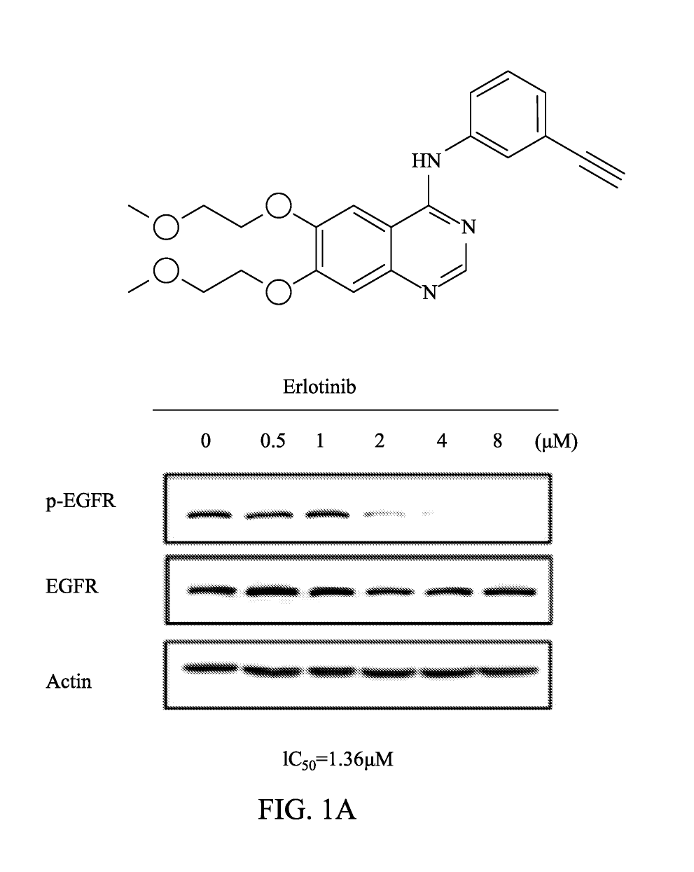

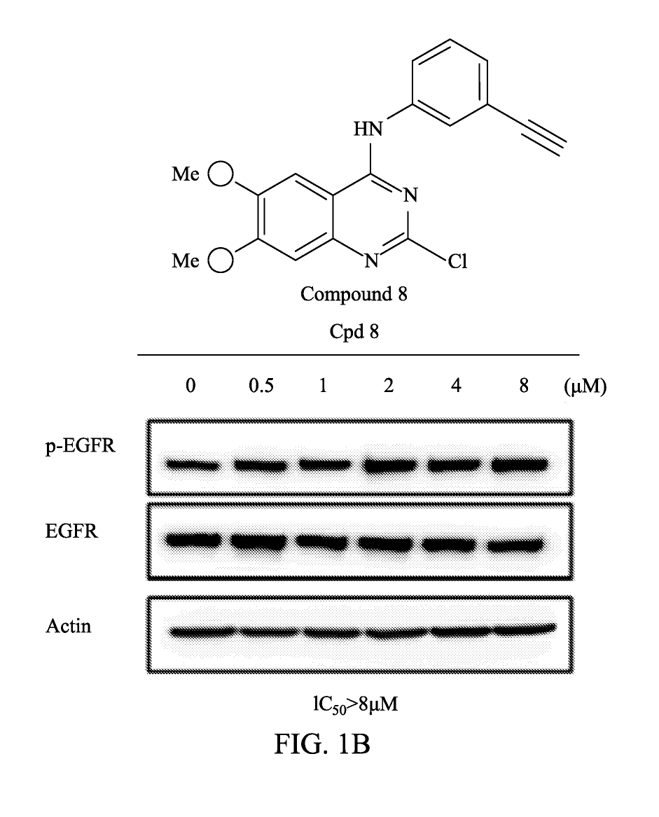

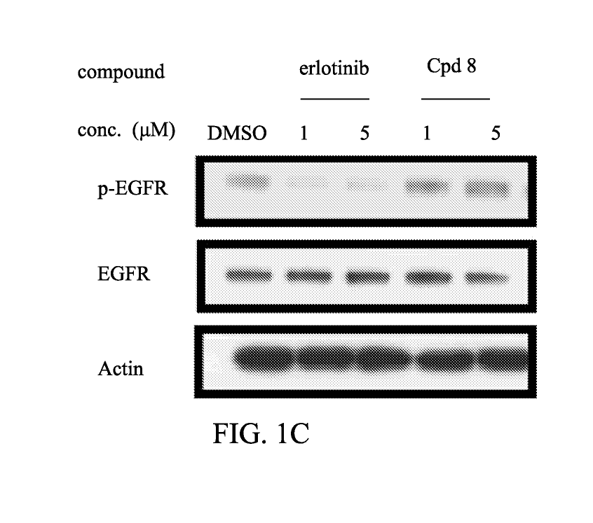

Some quinazoline derivatives having amino substitutes at position 4, such as erlotinb, gefitinib, and lapatinib, have been approved for clinical use in cancer patients. The chemical structure of compound 8 is similar to erlotinb (FIG. 1). A chloride atom at the 2-position of the quinazoline ring was further introduced to prevent the formation of hydrogen bonds between nitrogen atoms of compound 8 and T790 and M793 of EGFR kinase. A comparison of inhibiting the EGFR kinase activity by erlotinib and compound 8 in PC9 cells (a human lung adenocarcinoma) and H358 cells (a lung cancer cell line) was made by western blot.

PC9 cells (3×10⁵ cells) were treated with erlotinb or compound 8 at 0.5, 1, 2, 4, and 8 μM in 60 mm dishes for 24 hours. 40 μg/per lane of cell lysates were analyzed by western blot. The antibodies of actin, EGFR, and p-EGFR were from Cell Signaling (Danvers, Mass.). The results of western blot are shown in FIGS. 1A-1B. In FIGS. 1A-1B, actin lines are used to show an internal standard for the loading control in this western blot. The result of FIG. 1A shows that erlotinib was able to inhibit the phosphorylation of EGFR with IC₅₀ at 1.36 μM, but the result of FIG. 1B shows that compound 8 was devoid of EGFR kinase inhibition.

H358 cells were exposed to erlotinib or compound 8 at 1, and 5 μM for 24 hours and cell lysates were analyzed for EGFR phosphorylation. The result is shown in FIG. 1C. The result of FIG. 1C also shows that erlotinib was able to inhibit the phosphorylation of EGFR, but compound 8 was devoid of EGFR kinase inhibition.

The results above suggested that functional group connected to 2-position of quinazoline ring impeded nitrogen atom of quinazoline to act as a hydrogen acceptor and break the binding with EGFR. Therefore, a series of quinazoline derivatives (compounds 8-17) having a substituent at the 2 position of the quinazoline skeleton were synthesized. Another series of quinazoline derivatives (compounds 18-33) having various phenyl amine substituents at the position 2 of quinazoline were further synthesized. Moreover, the quinazoline skeleton was further simplified by using pyrimidine skeleton instead (compounds 1-7). The bioactivity of these compounds were analyzed and described below.

### Structure Activity Relationship of Pyrimidine and Quinazoline Derivatives

Pyrimidine derivatives (compounds 1-7) and quinazoline derivatives (compounds 8-24) were screened against a panel of SK-Hep-1 cell lines (a hepatocellular carcinoma (HCC) cell) for growth-inhibitory activities. MTT assay was used to measure growth inhibition. The compound concentrations causing 50% cell growth inhibition (IC₅₀ values) were determined by interpolation from dose-response curves. MTT (3-(4,5-dimethylthiazol-2-yl)-2,5-diphenyl tetrazolium bromide) assay was performed as follow.

The effect of individual test compounds on cell viability was assessed by using the MTT in 6 replicates. Gk-Hep-1 cells were seeded and incubated in 96-well, flat-bottomed plates for 24 hours, and were exposed to various concentrations of test compounds dissolved in DMSO (final concentration, 0.1%) for 48 hours. Controls received DMSO vehicle at a concentration equal to that in drug-treated cells. The medium was removed, replaced by 200 μL of 0.5 mg/mL MTT in 10% fetal bovine serum containing DMEM (Dulbecco's Modified Eagle Medium), and cells were incubated in the carbon dioxide incubator at 37° C. for 2 hours. Supernatants were removed from the wells and the reduced MTT dye was solubilized in 100 μL/well DMSO. Absorbance at 570 nm was determined on a plate reader.

The result of MTT assay showed that IC₅₀ values of compounds 6, 8, 13, and 18-23 were smaller than 10 μM. Especially, compounds 19 and 22 had the smallest IC₅₀ value (2.8±0.1 μM) among these compounds. Next. IC₅₀ values of compounds 7, 9, 10, 14, 17, and 24 were within 10-20 μM. As for compounds 1-5, 11, 12, 15, 16, the IC₅₀ values were all greater than 40 μM.

For compounds 8 and 9, the IC₅₀ values were very close (7.5±0.5 v. 12.3±0.6). Compound 9 was synthesized by methylation of the substituted phenyl amine group at the position 4 of the quinazoline. This implies that hydrogen donor ability of the substituted phenyl amine group is not necessary for the induction of cell death. In addition, as methylation at the substituted phenyl amine group is known to dramatically reduce the binding ability of quinazoline with EGFR, compound 9 further provides a proof that induction of cell death of quinazoline derivatives is independent of EGFR inhibition.

For compounds 11 and 15, a hydroxyl group was introduced into the phenyl ring. The IC₅₀ greater than 40 μM shows that no activity against SK-Hep1 cells. This suggests that hydrophobic interaction is required in this area.

For compounds 14 and 16, a phenyloxy group and a 4-cyano-phenyloxy was introduced into the phenyl ring. Compound 14 exhibited higher activity than compound 16 (15.3±0.6 μM v. >40 μM). This reveals that an electron-withdrawing group on the benzene ring is not favored for inducing cell death.

For the mono-substituted quinazoline derivatives (Embodiment 3), compound 8 exhibited the most potent growth inhibitory activity (7.5±0.5 μM). Interestingly, when the quinazoline skeleton of the mono-substituted quinazoline derivatives was simplified to the pyrimidine skeleton (Embodiment 1), no inhibitions were detected in cell growth assays. However, the compounds 6 and 7 with phenylamine di-substituents at positions 2, 4 in pyrimidine (Embodiment 2) showed more potent anti-tumor activity than the mono-substituted pyrimidine derivatives (Embodiment 1) in cell growth assays. This result suggests that a phenylamine group connected to position 2 of pyrimidine plays a crucial role in cancer-cell growth inhibitory activity.

Accordingly, a second substituted phenyl amine group was further introduced at the position 2 of the quinazoline (Embodiment 4). These derivatives showed more potent activity than mono-substituted derivatives against HCC cells. This result suggests that the second substituted phenyl amine group at the position 2 of the quinazoline plays a significant role in the cancer-cell growth inhibitory activity.

In compounds 18-24, compounds 19 and 22 exhibited higher potency with low IC₅₀ values (2.8 and 2.8 μM, respectively) against HCC cells whereas compound 24 only showed moderate activity (14.5 μM), indicating that substitutions with hydrophobic properties, such as phenyloxy (compound 19) and benzyl groups (compound 22) exhibited higher CIP2A inhibitory activity than the hydrophilic cyanophenyl groups (compound 24). In addition, compound 23 showed much better inhibition than compound 24 (3.9 μM v. 14.5 μM), suggesting that the connection position of cyanophenyl group to phenyl ring plays an important role in CIP2A inhibition.

### Validation of the Action Mode of Pyrimidine and Quinazoline Derivatives

### Inhibition of CIP2A Expression

Quinazoline derivatives have previously been evaluated as EGFR inhibitors. However, the quinazoline derivatives above had very low potency against EGFR because of the second substituted group at the position 2 of the quinazoline. However, the quinazoline derivatives above were found to be capable of repressing oncoprotein CIP2A expression and induced cell death as shown above. Therefore, it was hypothesized that quinazoline derivatives downregulate CIP2A and p-Akt, and consequently enhance cell apoptosis.

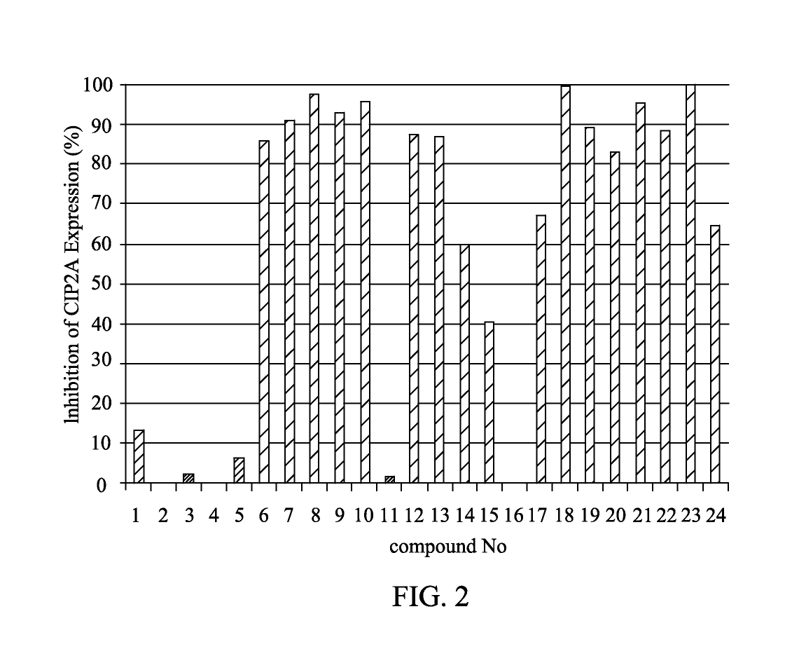

Accordingly, the pyrimidine and quinazoline derivatives above were screened by using western blot analysis for expression of CIP2A in SK-Hep1 cells. Before the western blot analysis, the SK-Hep-1 cells were maintained in DMEM supplemented with 10% FBS, 100 units/mL penicillin G, 100 μg/mL streptomycin sulfate and 25 μg/mL amphotericin B in a 37° C. humidified incubator in an atmosphere of 5% CO₂ in air. In this western blot analysis. SK-Hep1 cells were respectively treated with compounds 1-24 at a concentration of 20 μM for 24 hours. The results of inhibition effect on CIP2A expression are shown in FIG. 2. In FIG. 2, the mono-substituted pyrimidine derivatives (compounds 1-5) have no appreciable change in CIP2A expression. Di-substituted pyrimidine (compounds 6-7) and quinazoline compounds (compounds 8-24) on the other hand showed a high degree of repression of CIP2A.

Quantitative Polymerase Chain Reaction (qPCR) Assay

In addition, a quantitative polymerase chain reaction (qPCR) assay was used to find the correlation coefficient (R²) between the IC₅₀ of CIP2A inhibition and the IC₅₀ of cell growth. The correlation coefficient (R²) between the IC₅₀ of CIP2A inhibition and the IC₅₀ of cell growth was found to be 0.9519. This indicates that the decreased level of CIP2A induced by these derivatives is well correlated with cell toxicity.

The qPCR was performed as follow. Total RNA was isolated from SK-Hep1 cell line with TRIzol (Invitrogen). An aliquot of 2.5 μg/12.1 μL of total RNA was used as the template in the synthesis of first-strand cDNA using an oligo(dT) primer and the AMV reverse transcriptase system (Roche Diagnostics) by Thermal Cycler (RTC-200, MJ Research). The method of qPCR was followed according to the method described by Ponchel et al (Ponchel, F. et al. BMC Biotechnol 2003, 3, 18). qPCR was performed using a Roche Light Cycler 480 sequence detection system (Roche Applied Science,). Thermocycling was performed in a final volume of 20 μl containing 2.5 μl of cDNA sample, 200 nM of each of the primers, and 6.5 μL of SYBR Green I master mix (Roche).

The relative differences in expression levels between genes were expressed using cycle time (Ct) values as follows: the Ct value of the gene of CIP2A was first normalized to that for GAPDH in the same sample, then the difference between the treatment and control group was calculated and expressed as an increase or decrease in cycle numbers compared with the control. Oligonucleotide sequences were as follows: CIP2A, 5′-TGG CAA GAT TGA CCT GGG ATT TGG A-3′(sense) and 5′-AGG AGT AAT CAA ACG TGG GTC CTG A-3′(antisense); GAPDH, 5′-CGA CCA CTT TGT CAA GCT CA-3′(sense) and 5′-AGG GGT CIA CAT GGC AAC TG-3′ (antisense). The following PCR conditions were used: denaturation at 95° C. for 10 min followed by 40 cycles of 94° C. for 1 min, annealing for 1 min at 60° C., and elongation for 1 min at 72° C., and a final elongation step at 72° C. for 10 min.

Correlation of Down-Regulating CIP2A and P-Akt with EGFR Phosphorylation

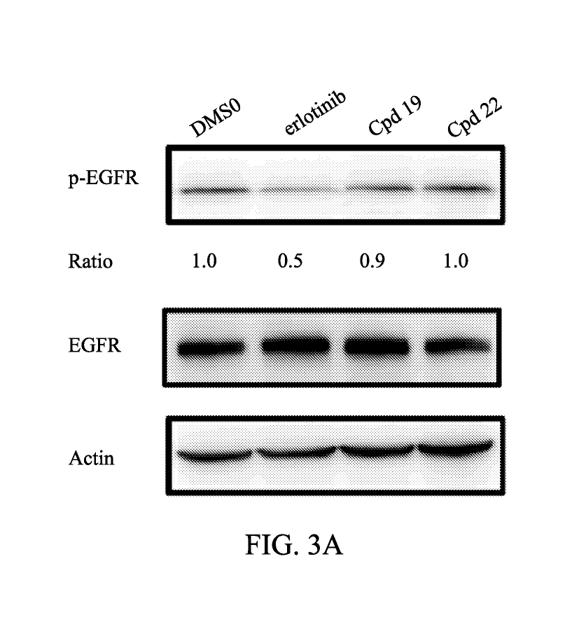

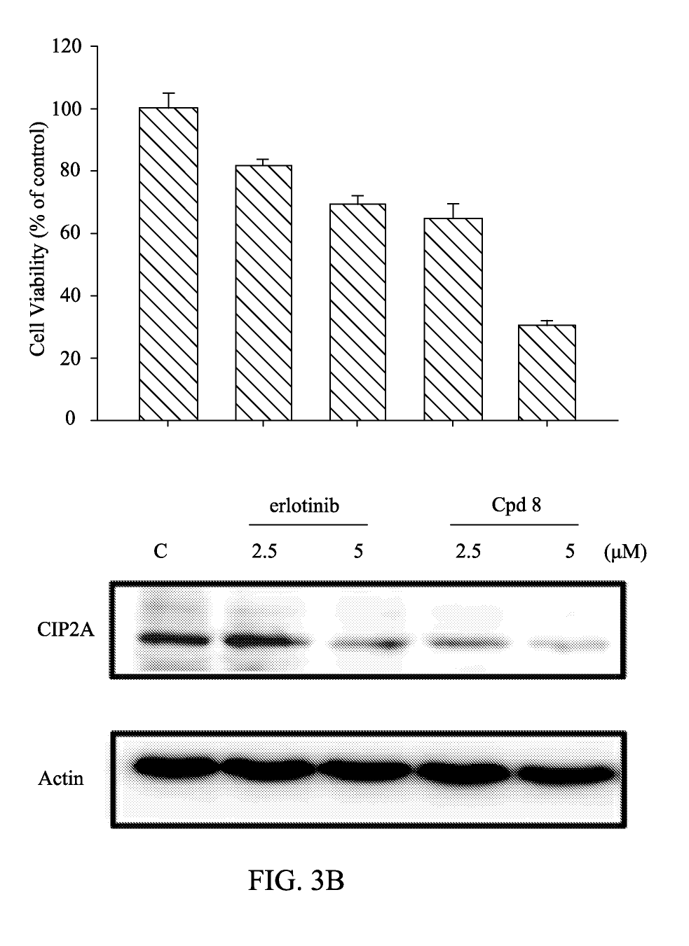

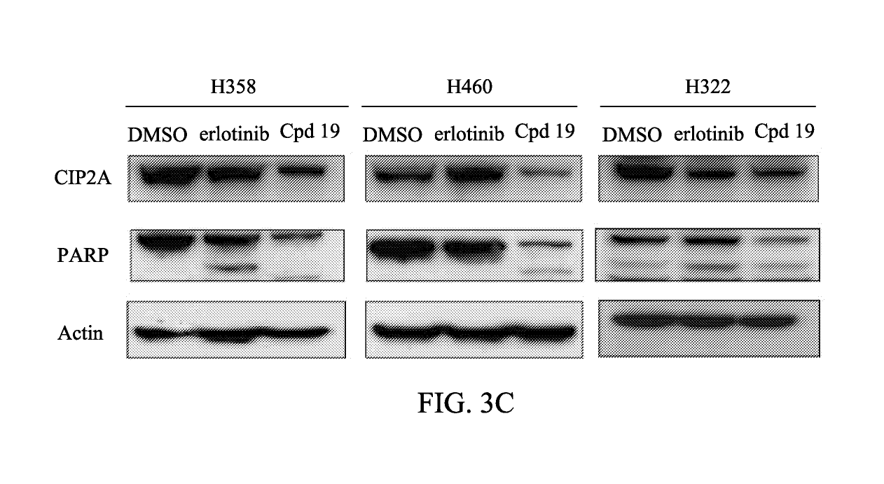

Next, the most potent compounds 19 and 22 were used to study whether down-regulation CIP2A and p-Akt is correlated to EGFR phosphorylation. FIG. 3A is the western blot analysis of inhibiting EGFR phosphorylation activity by erlotinib, compound 19, and compound 22, respectively. In FIG. 3A, PC9 cells (a human lung adenocarcinoma) were treated with erlotinib, compound 19, and compound 22 at 2 μM for 24 h. The result of FIG. 3A shows that compound 19 and 22 have no inhibitory effect on EGFR kinase. This data confirms that the second substituted-quinazoline derivatives significantly reduce the binding affinity to ATP bind domain of EGFR kinase.

Cell viability, measured by MIT assay, and CIP2A expressions, analyzed by western blot, in response to compound 19 or erlotinib treatment in SK-Hep1 cell line were analyzed. SK-Hep-1 cells were treated with erlotinib and compound 19 at 2.5 and 5 μM for 24 h, respectively. The SK-Hep1 cells were then analyzed with western blot assay and MTT assay. The results are shown in FIG. 3B. In FIG. 3B, compound 19 can reduce the CIP2A expression and the cell viability in a dosage dependent manner, and was more potent than erlotinib. These results suggest that CIP2A plays an important role in regulating cell viability.

Similarly, CIP2A expressions analyzed by western blot were also performed for lung cancer cells, H358, H460, and H322 cell lines. Moreover, drug-induced apoptotic cell death was also assessed by western blot analysis of activated caspases cleaved poly(ADP-ribose) polymerase (PARP). The cleavage of PARP is explained as follow. The compound induces apoptotic signal which cleave the procaspase 3 to the active caspase 3. The activation of caspase 3 further cleaves PARP and inactivate PAPR function. The events are thought to be required in late apoptosis. Therefore, the PARP cleavage is an indicator of apoptosis.

The results are shown in FIG. 3C. The result of FIG. 3C shows that compound 19 can down-regulate the expression of CIP2A and induce the cleavage of PARP in all of the H358. H460, and H322 cell lines. In the PARP row of FIG. 3C, the upper bands represented the uncleavage PARP, and the middle band and lower band represented cleavage PARP.

Correlation Between Down-Regulating CIP2A and Suppressing p-Akt

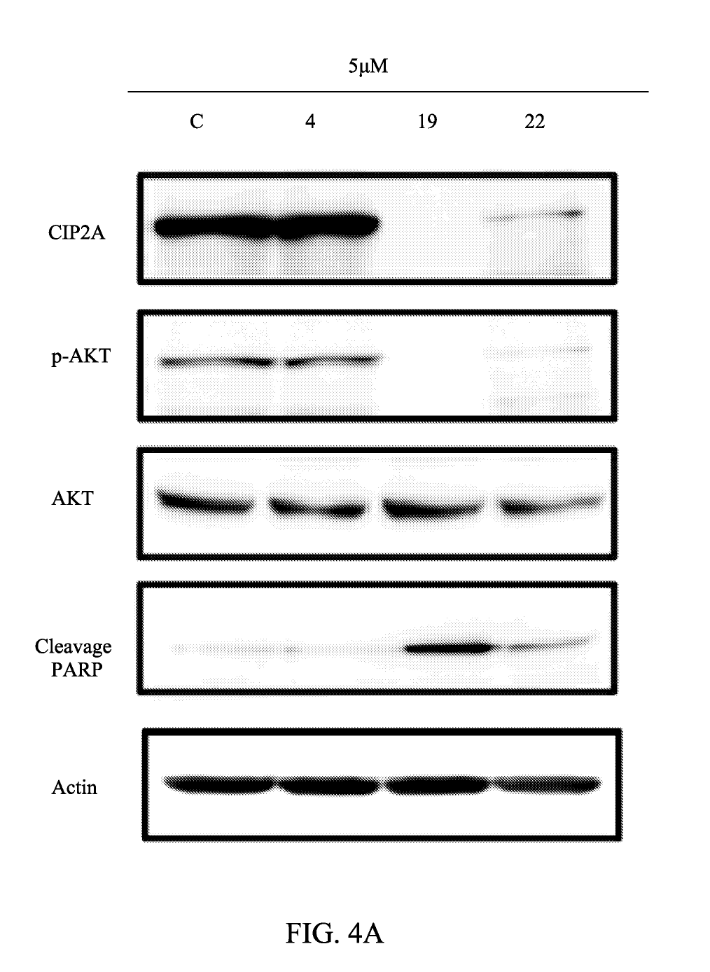

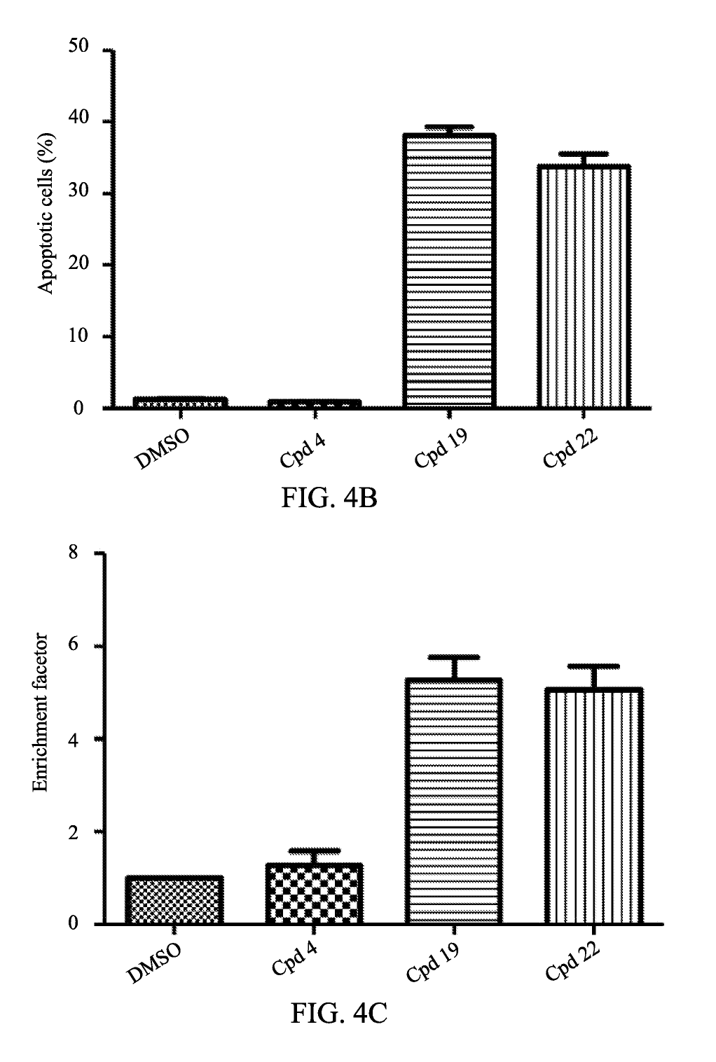

Next, compounds 11, 19, and 22 were used to explore whether downregulation of CIP2A lead to suppression of p-Akt. FIG. 4A is the western blot analysis of the effect of the compounds 4, 19, and 22 on phosphorylation of Akt. PARP and Actin. In this western blot analysis, SK-Hep-1 cells were respectively treated with compounds 4, 19, and 22 at a concentration of 5 μM for 30 hours. In FIG. 4A, compounds 19 and 22 showed the activity of inhibiting CIP2A expression, reducing p-Akt level, and inducing PARP cleavage. That is, compound 19 and 22 can substantially increase apoptotic cell death to inhibit cancer growth. However, compound 4 showed no activity of inhibiting CIP2A expression, reducing p-Akt level, and inducing PARP cleavage.

FIG. 4B is flow cytometry analysis of cell death induced by compounds 4, 19, and 22 at 5 μM, after 24 h of treatment in SK-Hep-1 cells. The apoptotic cells were assessed by flow cytometry (sub-G1). After Sk-Hep-1 cells were treated with compounds 4, 19, and 22. Sk-Hep-1 cells were trypsinized, collected by centrifugation and resuspended in PBS. After centrifugation, the cells were washed in PBS and resuspended in potassium iodide (PI) staining solution. Specimens were incubated in the dark for 30 min at 37° C. and then analyzed with an EPICS Profile II flow cytometer (Coulter Corp., Hialeah, Fla.). All experiments were performed in triplicate

FIG. 4C is ELISA analysis of cell death to analyze effects of compounds 4, 19, and 22 on DNA fragmentation in SK-Hep-1 cells. The effect of compounds 4, 19 and 22 on cell viability was assessed by a cell death detection ELISA kit (Roche Applied Science, Mannheim, Germany). SK-Hep-1 cells were treated with compounds 4 and 19 at 22 at 2.5 and 5 μM for 24 h. The cells were collected and assayed according to the standard protocol provided by the manufacturer.

The results of FIGS. 4B-4C show that compounds 19 and 22 induced cell apoptosis. These results are consistent with their inhibition of CIP2A expression.

### CIP2A Knockdown Experiment (in Supplementary Material)

In order to know whether CIP2A is a key regulator of cell survival, we have used genetic knockdown CIP2A and than determine the cell survival with colongenic assay.

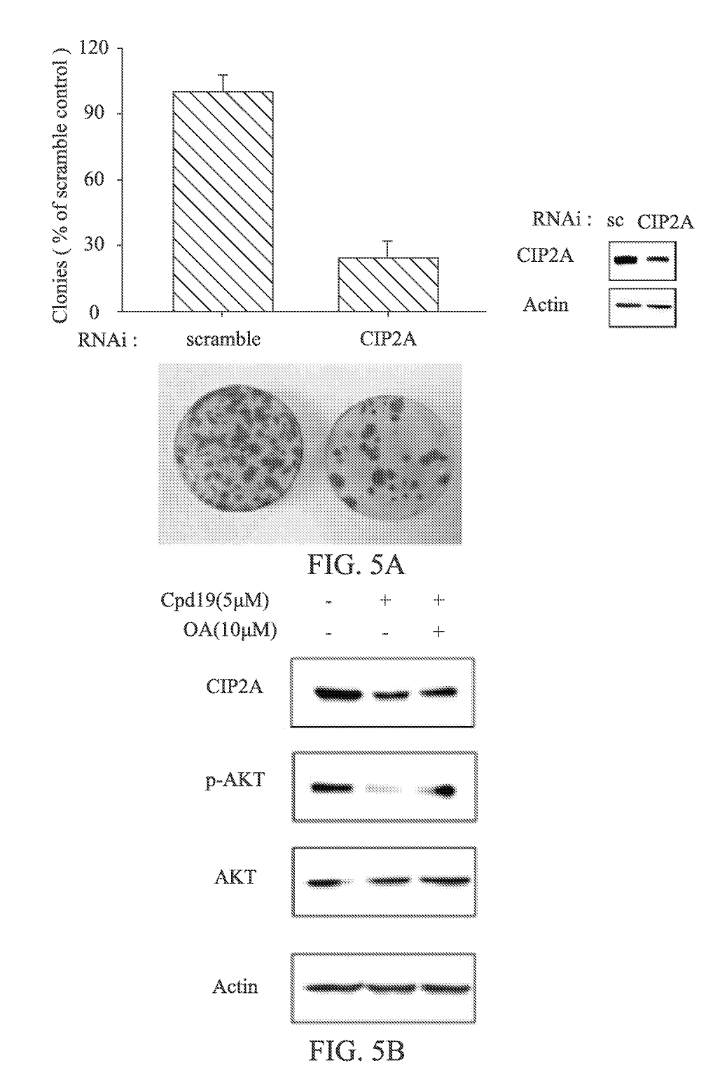

For colony formation, SK-Hep1 cells transfected with scramble siRNA or CIP2A-specific siRNA for 48 hours were seeded in triplicate onto 6 cm plates (10,000 cells per plate). After 7 days of culturing, cells were stained with crystal violet, and colonies containing more than 50 cells were counted. The obtained results are shown in FIG. 5A.

The effect of okadaic acid (OA) in compound 19 induced CIP2A inhibition is shown in FIG. 5B. In FIG. 5B, it can be seen that okadaic acid, a phosphatase inhibitor, reversed p-Akt level in compound 19 treated SK-Hep-1 cells, indicating that compound 19 inhibit CIP2A, activate PP2A and further dephosphorelate p-Akt. In other words, in the present of okadaic acid, the activation of PP2A by compound 19-induced CIP2A inactivation is blocked.

### Animal Test

In vivo efficacy was determined in nude mice with PLC5 and Huh-7 xenografts.

### Xenograft Tumor Growth

Male NCr athymic nude mice (5-7 weeks of age) were obtained from the National Laboratory Animal Center (Taipei, Taiwan). The mice were housed in groups and maintained under standard laboratory conditions on a 12-hour light-dark cycle. They were given access to sterilized food and water ad libitum. All experimental procedures using these mice were performed in accordance with protocols approved by the Institutional Laboratory Animal Care and Use Committee of National Taiwan University. Each mouse was inoculated s.c. in the dorsal flank with 1×10⁶ HCC cells suspended in 0.1 ml of serum-free medium containing 50% Matrigel (BD Biosciences, Bedford, Mass.). When tumors reached 150-200 mm³, mice received erlotinib at 50 mg/kg/day or compounds 8 and 19 at 20 mg/kg/day daily by oral gavage for 3 weeks. Controls received vehicle.

### Statistical Analysis

Tumor growth data points are reported as mean tumor volume±SE. Comparisons of mean values were performed using the independent samples t test in SPSS for Windows 11.5 software (SPSS, Inc., Chicago, Ill.).

### Cell Culture of PLC5 and Huh-7 HCC Cell Lines (HCC Cell Lines)

PLC5 HCC cell line was obtained from American Type Culture Collection (ATCC; Manassas, Va.). The Huh-7 HCC cell line was obtained from the Health Science Research Resources Bank (HSRRB; Osaka, Japan; JCRB0403).

All cells were immediately expanded and frozen down such that all cell lines could be restarted every 3 months from a frozen vial of the same batch of cells. No further authentication was conducted in our lab. Cells were maintained in DMEM supplemented with 10% FBS, 100 units/mL penicillin G, 100 μg/mL streptomycin sulfate, and 25 μg/mL amphotericin B in a 37° C. humidified incubator and an atmosphere of 5% CO₂ in air. Lysates of HCC cells treated with drugs at the indicated concentrations for various periods of time were prepared for immunoblotting of PARP, P-Akt, Akt, etc.

### In Vivo Effects of Erlotinib and Compounds 8 and 19 on HCC Xenograft Tumors

The in vivo effects of erlotinib, compound 8, and compound 19 on HCC xenograft tumors were analyzed. Tumor-bearing mice were treated with vehicle, erlotinib (50 mg/kg/day), compound 8 (20 mg/kg/day), or compound 19 (20 mg/kg/day) p.o. daily for 3 weeks. All animals tolerated the treatments well without observable signs of toxicity and had stable body weights throughout the course of study. No gross pathologic abnormalities were noted at necropsy.

The results of xenograft study above were listed in Table below. The data listed in the Table below were the data after 21 days of treatment.

| | Erlotinib | Cpd 8 | Cpd19 |
|---|---|---|---|
| | (inhibition) | (inhibition) | (inhibition) |
| PLC5 cell | 50.0% | 45.7% | 60.6% |
| Huh7 cell | 8.8% | 40.3% | 34.8% |

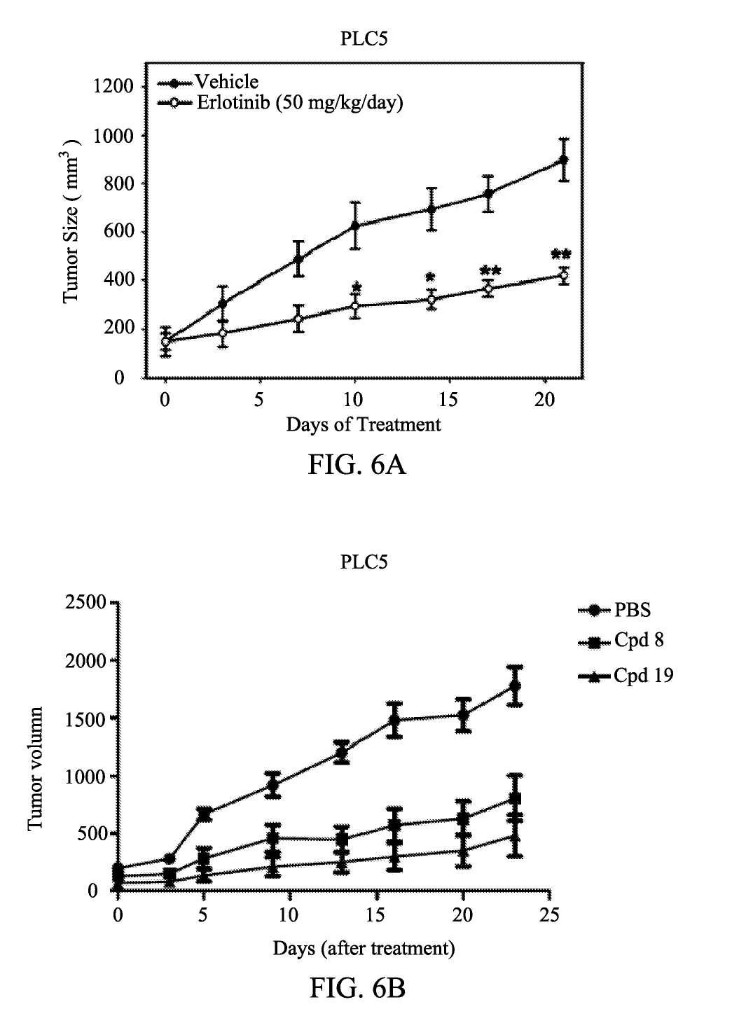

FIGS. 6A and 6B shows the tumor size changed with days of treatment by erlotinib, compound 8, and compound 19 in PLC5 xenograft tumor. The result of FIG. 5B shows that compounds 8 and 19 can significantly reduced the growth of tumor in sensitive PLC5 tumors. Especially, compound 19 even shown better inhibitory effect on PLC5 tumors than erlotinib (FIG. 5A).

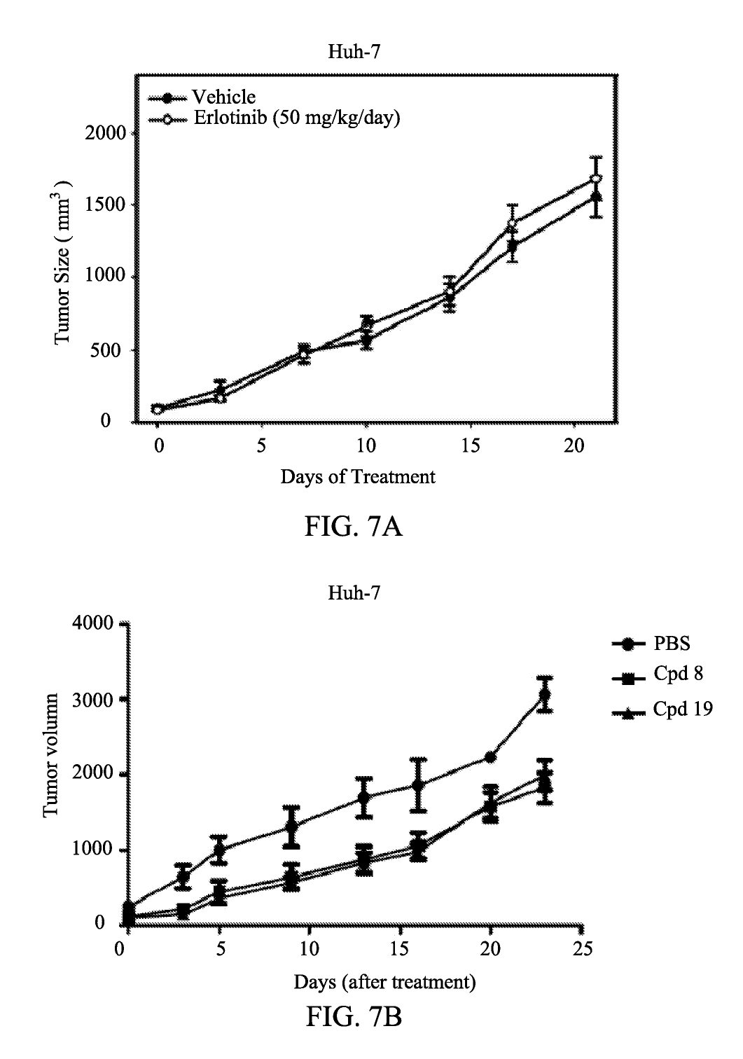

FIGS. 7A and 7B shows the tumor size changed with days of treatment by erlotinib, compound 8, and compound 19 in Huh-7 xenograft tumor. The result of FIG. 6A shows that erlotinib treatment had no effect on tumor growth in resistant Huh-7 cells. However, the result of FIG. 6B shows that compounds 8 and 19 still possess better inhibitory effect on Huh-7 tumors than erlotinib.

In light of foregoing, a series of pyrimidine and quinazoline-derived compounds were synthesized and their cytotoxicity was explored with interesting SAR results. Structural modifications indicated that di-phenylamine derivatives with quinazoline and pyrimidine skeletons are required for activity.

According to MTT assay, most of these derivatives had micromolar level potency against SK-Hep-1 cells. Compounds 19 and 22 showed the most potent inhibition of CIP2A expression and cell survival activity, whereas compound 4 had no activity in either assay. Furthermore, compounds 19 and 22 reduced Akt phosphorylation after repressing CIP2A, whereas compound 4 had no activity against p-Akt and CIP2A.

These results suggest selective sensitivity in response to the different substituted functional groups in quinazoline. Moreover inhibition of CIP2A expression correlated with cytotoxicity in SK-Hep-1 cells upon drug treatment. Testing of compounds 19 and 22 in an in vivo HCC model shows that compounds 19 and 22 are capable of significantly reducing the growth of tumor in sensitive PLC5 tumors.

All the features disclosed in this specification (including any accompanying claims, abstract, and drawings) may be replaced by alternative features serving the same, equivalent or similar purpose, unless expressly stated otherwise. Thus, each feature disclosed is one example only of a generic series of equivalent or similar features.

What is claimed is:

1. An aryl amine substituted pyrimidine having a chemical structure (I) or (II) below: 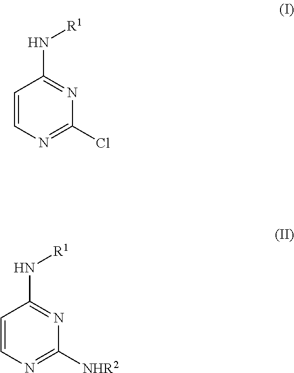
wherein R¹ and R² are same or different substituted phenyl groups, and the substituted phenyl group each is

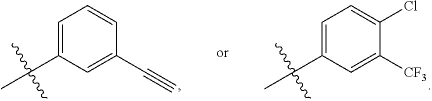

2. An aryl amine substituted quinazoline having a chemical structure (III) or (IV) below: 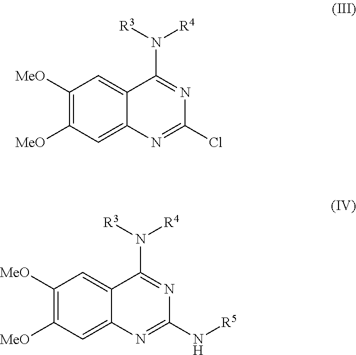
wherein R³ is an aliphatic-substituted phenyl group, a halo-substituted phenyl group, a hydroxyl-substituted phenyl group, or an aryloxy-substituted phenyl group;
R⁴ is H, an aliphatic group with carbon number of 1-5, an amino-substituted aliphatic group, or a benzyl group;
R⁵ is aliphatic substituted phenyl group, a halo-substituted phenyl group, an aryloxy-substituted phenyl group, a benzyl group, a halo substituted benzyl group, an alkoxy substituted phenyl group, an arylamino-substituted phenyl group, an amidyl-substituted phenyl group, an ArO(CO)NH-substituted phenyl group, or Ph-SO₂—NH-substituted phenyl group.

3. The aryl amine substituted quinazoline of claim 2, wherein the R³ is

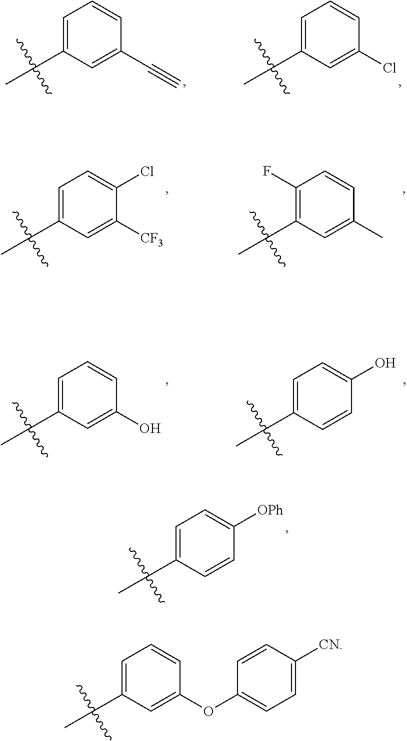

4. The aryl amine substituted quinazoline of claim 2, wherein the R⁴ is H, Me

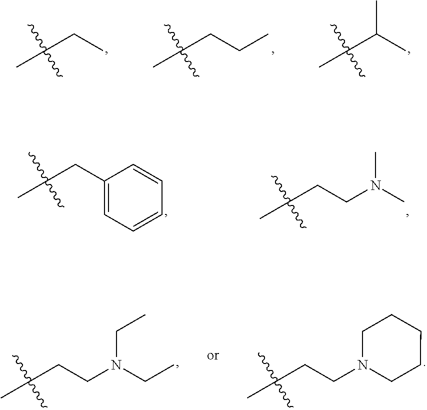

5. The aryl amine substituted quinazoline of claim 2, wherein the R⁵ is

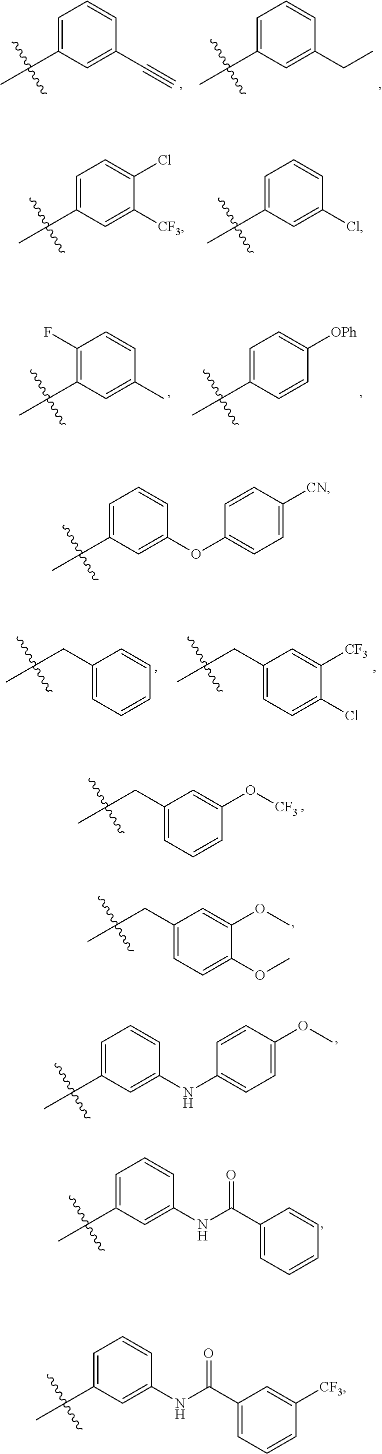

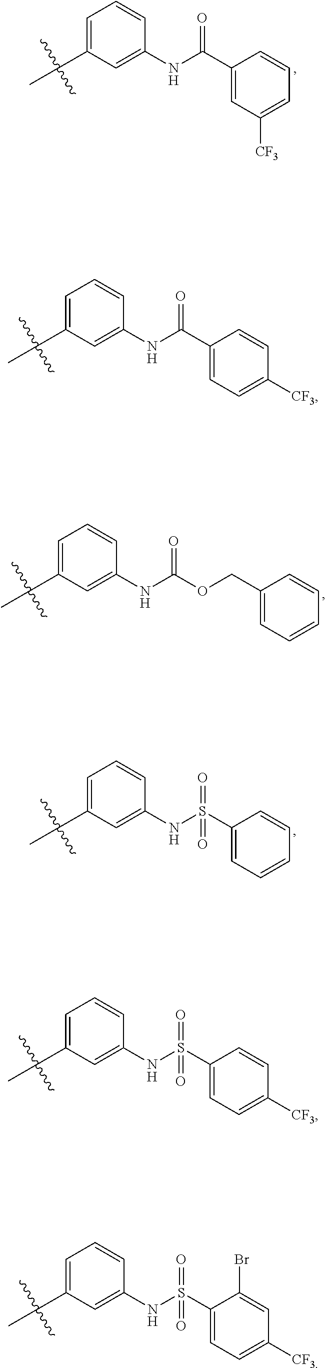 [SMILES: *c1cccc(NC(=O)c2cccc(C(F)(F)F)c2)c1 ; *c1cccc(NC(=O)c2ccc(C(F)(F)F)cc2)c1 ; *c1cccc(NC(=O)OCc2ccccc2)c1 ; *c1cccc(NS(=O)(=O)c2ccccc2)c1 ; *c1cccc(NS(=O)(=O)c2ccc(C(F)(F)F)cc2)c1 ; *c1cccc(NS(=O)(=O)c2ccc(C(F)(F)F)cc2Br)c1]

6. A method of synthesizing the aryl amine substituted pyrimidine of claim 1, comprising:
reacting 2,4-dichloropyrimidine with a first substituted phenyl amine to obtain the compound of the chemical structure (I), wherein the first substituted phenyl amine having a first substituted phenyl group of

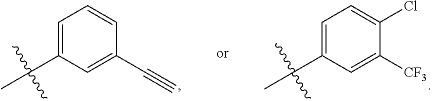

7. The method of claim 3, further comprising reacting the compound of the chemical structure (I) with a second substituted phenyl amine to obtain the compound of the chemical structure (II), wherein the second substituted phenyl amine having a second substituted phenyl group of

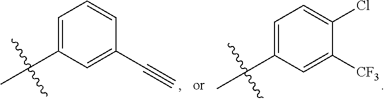

8. A method of synthesizing the aryl amine substituted quinazoline having the chemical structure (III) of claim 2, comprising:
reacting 2,4-dichloroquinazoline with a substituted phenyl amine, wherein the substituted phenyl amine having a substituted phenyl group of

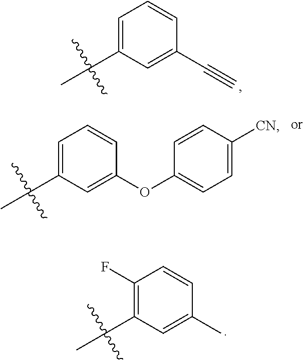

9. A method of synthesizing the aryl amine substituted quinazoline having the chemical structure (IV) of claim 2, comprising:
reacting 2,4-dichloroquinazoline with R³R⁴NH to obtain 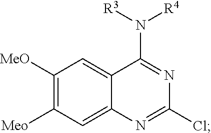
and
reacting 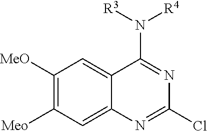
with a R⁵NH₂.

10. A pharmaceutical composition comprising:
an effective amount of a compound having a chemical structure (I), (II), (III), or (IV) below: 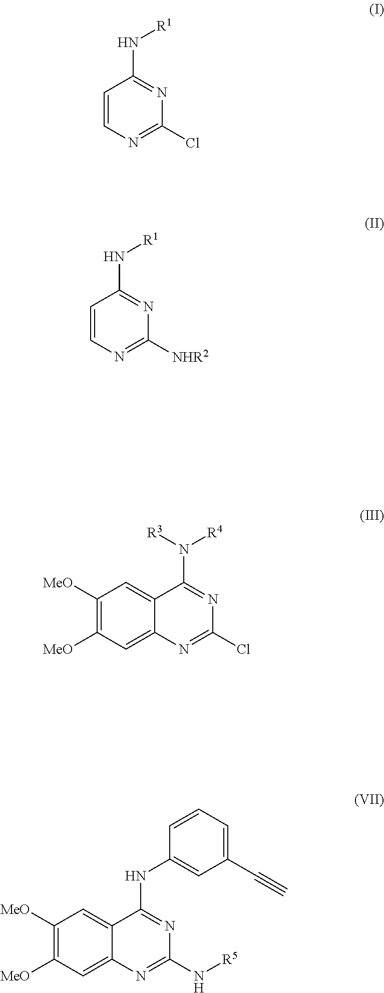
wherein R¹ and R² are same or different substituted phenyl groups, and the substituted phenyl group each is 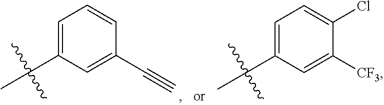
R³ is 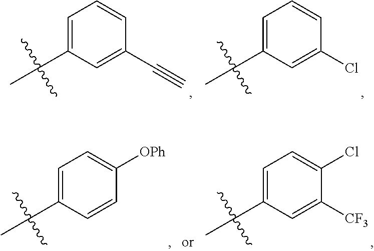
R⁴ is H or methyl group, and
R⁵ is 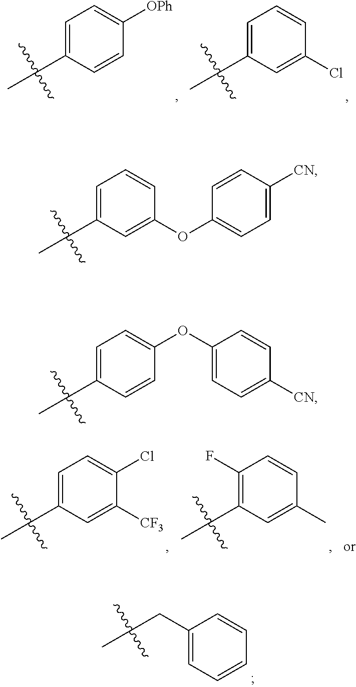
and
a pharmaceutically acceptable carrier.

11. The pharmaceutical composition of claim 10, wherein the compound having a chemical structure (V) or (VI) below.

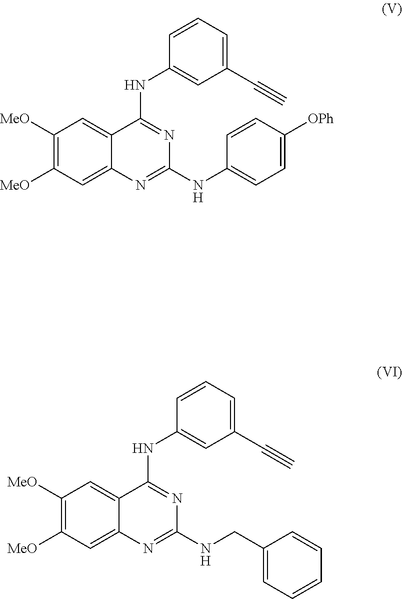 [SMILES: C#Cc1cccc(Nc2nc(Nc3ccc(Oc4ccccc4)cc3)nc3cc(OC)c(OC)cc23)c1 ; C#Cc1cccc(Nc2nc(NCc3ccccc3)nc3cc(OC)c(OC)cc23)c1]

12. A method of inhibiting the expression of cancerous inhibitor of protein phosphatase 2A (abbreviated as CIP2A), comprising:
contacting a cell with an effective amount of a compound having a chemical structure (I), (II), (III), or (VII) below: 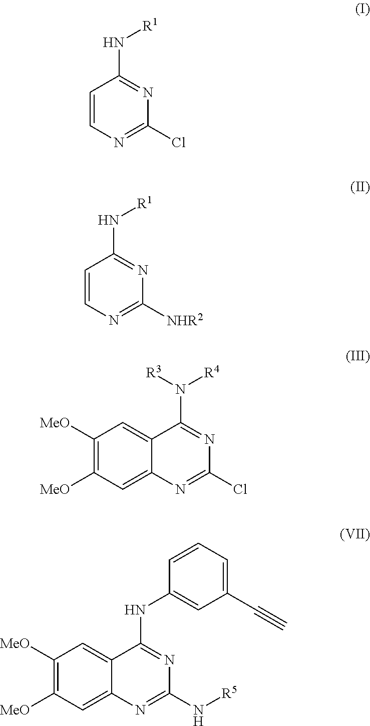
wherein R¹ and R² are same or different substituted phenyl groups, and the substituted phenyl group each is 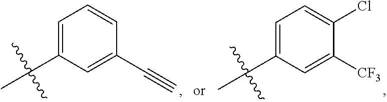
R³ is 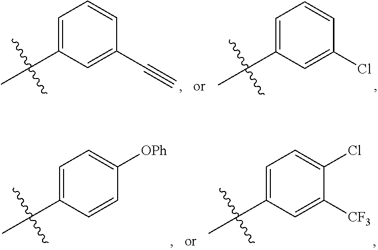
R⁴ is H or methyl group, and
R⁵ is

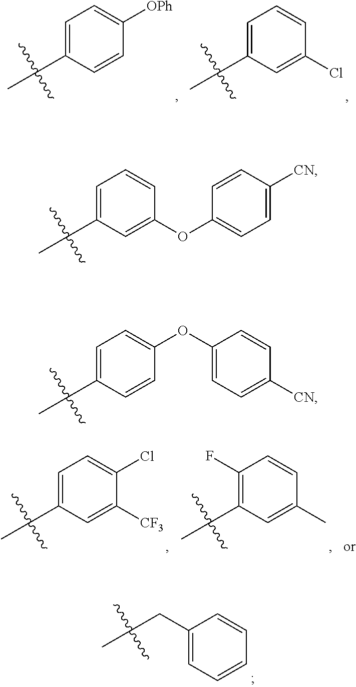

13. The method of claim 12, wherein the compound having a chemical structure (V) or (VI) below.

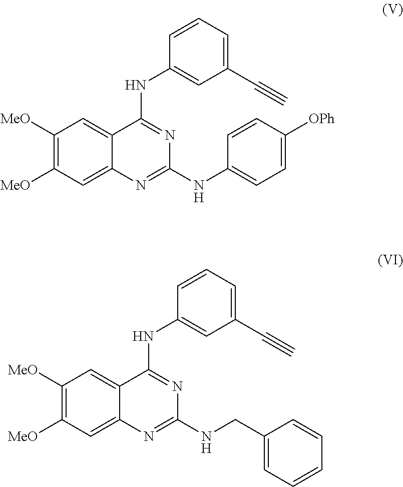 [SMILES: C#Cc1cccc(Nc2nc(Nc3ccc(Oc4ccccc4)cc3)nc3cc(OC)c(OC)cc23)c1 ; C#Cc1cccc(Nc2nc(NCc3ccccc3)nc3cc(OC)c(OC)cc23)c1]

14. A method of treating a cancer, comprising:
administrating an effective amount of a compound having a chemical structure (I), (II), (III), or (VII) below by a needed subject: 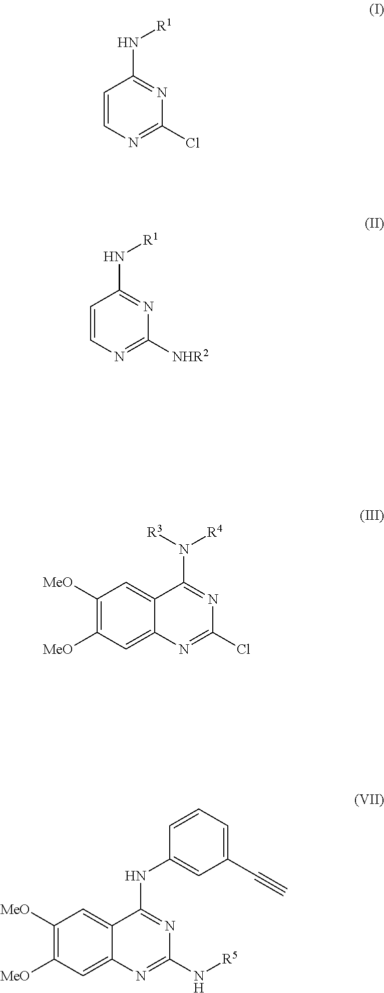
wherein R¹ and R² are same or different substituted phenyl groups, and the substituted phenyl group each is 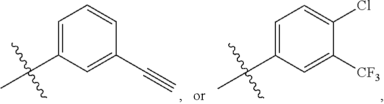
R³ is 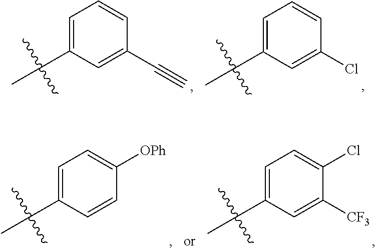
R⁴ is H or methyl group, and
R⁵ is

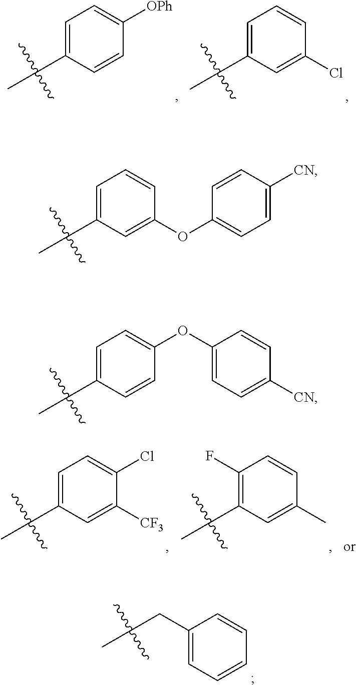

15. The method of claim 14, wherein the compound having a chemical structure (V) or (VI) below.

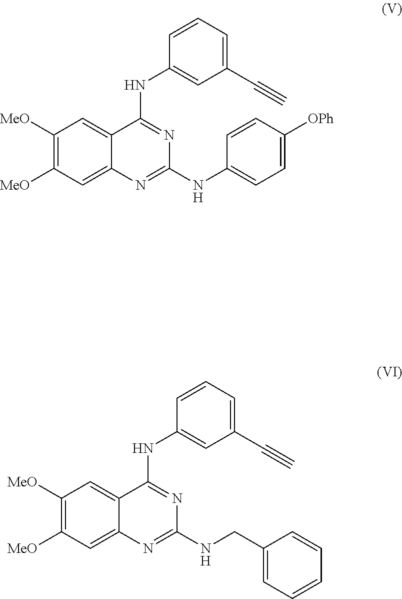 [SMILES: C#Cc1cccc(Nc2nc(Nc3ccc(Oc4ccccc4)cc3)nc3cc(OC)c(OC)cc23)c1 ; C#Cc1cccc(Nc2nc(NCc3ccccc3)nc3cc(OC)c(OC)cc23)c1]

16. The method of claim 14, wherein the cancer is a hepatocellular carcinoma or a lung cancer.
